(19) United States
(12) Patent Application Publication
(10) Pub. No.: US 2005/0282863 A1
(43) Pub. Date: Dec. 22, 2005

(54) Novel-N substituted dihydrobenzothiepino, dihydrobenzoxepino and tetrahydro benzocyclohepta indoles as selective estrogen receptor modulators

(75) Inventors: Kanchan Hajela, Lucknow (IN); Ashok Kumar Jha, Lucknow (IN); Man Mohan Singh, Lucknow (IN); Girish Kumar Jain, Lucknow (IN); Anil Kumar Balapure, Lucknow (IN); Anila Dwivedy, Lucknow (IN); Bharat Agarwal, Lucknow (IN); Puvvada Sri Ramchandra Murthy, Lucknow (IN)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Council of Scientific & Industrial Research, New Delhi (IN)

(21) Appl. No.: 11/089,907

(22) Filed: Mar. 25, 2005

Related U.S. Application Data
(60) Provisional application No. 60/558,350, filed on Mar. 31, 2004.

(51) Int. Cl. A61K 31/454; A61K 31/407; C07D 498/02

(52) U.S. Cl. 514/321; 514/410; 546/198; 548/421

(57) ABSTRACT

The invention provides a novel class of N-substituted dihydrobenzothiepino, dihydrobenzoxepino and tetrahydro benzocyclohepta indoles and their pharmaceutically acceptable salts, and methods for of synthesizing these compounds. The invention further comprises pharmaceutical compositions and methods of use for these compounds for the treatment of estrogen related diseases or disorders.

### CROSS-REFERENCE TO RELATED APPLICATIONS

This application claims the benefit of U.S. Provisional Application No. 60/558,350, filed Mar. 31, 2004; which is hereby incorporated by reference.

### FIELD OF THE INVENTION

This invention relates to a novel class of N-substituted dihydrobenzothiepino, dihydrobenzoxepino and tetrahydro benzocyclohepta indoles, their pharmaceutically acceptable salts showing selective estrogen receptor modulator activity.

The present invention further relates to the processes for the preparation of pharmaceutically active compounds, their pharmaceutically acceptable salts and compositions of the principal aspect of the present invention. The compounds are represented by the general structure I as shown below. 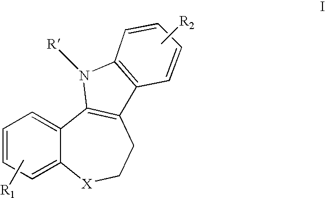
X is —O—, —S— or CH₂,
R′ is Y—(CH₂)ₙ or Y—(CH₂)ₙ—O-Ph
R₁ and R₂ are independently H, OH, —O(C₁-C₆ alkyl), —OCOC₆H₅, —OCO(C₁-C₆ alkyl), —OSO₂(C₄-C₆ alkyl), —OSO₂CF₃, Cl or F;
n is 2 through 6;
Y is selected from the moiety —NR₃R₄ wherein R₃ and R₄ are independently selected from groups consisting such as pyrrolidinoethyl, piperidinoethyl, dimethylaminoethyl, diethylaminoethyl, C₃-C₇ cycloalkyl;
a five membered saturated, unsaturated or partially unsaturated heterocycle containing up to two heteroatoms selected from the groups consisting of —O—, —NH—, —N(C₁C₄ alkyl)-, N═ and —S(O)ₘ, wherein m is an integer of from 0-2, optionally substituted with 1-3 substituents independently selected from the group consisting of H, OH, halo, nitro, cyano, SH, SO₂R₁, CO₂H, CONHR₁, NH₂;
a six membered saturated, unsaturated or partially unsaturated heterocycle containing up to two heteroatoms selected from the groups consisting of —O—, —NH—, —N(C₁C₄ alkyl)-, N═ and —S(O)ₘ, wherein m is an integer of from 0-2, optionally substituted with 1-3 substituents independently selected from the group consisting of H, OH, halo, nitro, cyano, SH, SO₂R₁, CO₂H, CONHR₁, NH₂;
a seven membered saturated, unsaturated or partially unsaturated heterocycle containing up to two heteroatoms selected from the groups consisting of —O—, —NH—, —N(C₁C₄ alkyl)-, N═ and —S(O)ₘ, wherein m is an integer of from 0-2, optionally substituted with 1-3 substituents independently selected from the group consisting of H, OH, halo, nitro, cyano, SH, SO₂R₁, CO₂H, CONHR₁, NH₂; a bicyclic heterocyclic containing from 6-12 carbon atoms either bridged or fused and containing up to two heteroatoms selected from the groups consisting of —O—, —NH—, —N(C₁C₄ alkyl)-, N═ and —S(O)ₘ, wherein m is an integer of from 0-2, optionally substituted with 1-3 substituents independently selected from the group consisting of H, OH, halo, nitro, cyano, SH, SO₂R₁, CO₂H, CONHR₁, NH₂.
   Within the scope of the compounds described above are the preferred compounds of formula I wherein R₁, R₂, R₃, R₄, X, n are as defined above and Y is selected from saturated five, six, seven membered heterocyclic or bicyclic structures with the heteroatoms and substituents described as above.

### BACKGROUND OF THE INVENTION

Clinical and epidemiological studies have shown that the post-menopausal state is characterised by changes in the level of circulating sex hormones, particularly the dramatic reduction in plasma levels of 17β-estradiol. This constitutes an important risk factor for various pathological conditions and is often referred to as Post-menopausal Syndrome. Osteopenia that accompanies menopause continues to represent a major public health problem in women health care. It poses risk to quality of life during old age and, if left unchecked, the cumulative loss of bone can potentially compromise the skeletons structural integrity resulting in osteoporosis.

Post-menopausal osteoporosis is a disorder characterised by a reduction in bone mass per unit volume with an alteration in bone microarchitecture that results in an increased susceptibility to fractures. Osteoporosis is now recognised as one of the most important disorders of aging. The most vulnerable tissue in the bone to the effects of post-menopausal osteoporosis is the trabecular bone. This tissue is often referred to as spongy or cancellous bone present near the joints and in the vertebrae of the spine. Most women lose from about 20% to 60% of bone mass in trabecular region of the bone within 3 to 6 years after menopause that leads to failure and fracture of bone. The most common fractures are those associated with bones which are highly dependent on the trabecular support, for example the vertebrae, the neck, and the weight bearing bone such as the femur and the forearm. Indeed the hip fracture, collie's fractures and vertebral crush fractures are also the hallmarks of post-menopausal osteoporosis. This rapid loss of bone occurs as a result of increased bone resorption relative to bone formation or a direct consequence of an imbalance between osteoclastic and osteoblastic activity coupled with an increased rate of bone turnover observed with menopause. Major efforts to reduce the risk and incidence of fractures have focused on the development of compounds/therapies that conserve skeletal mass by inhibiting bone resorption and/or by enhancing bone formation (Masahiko Sato, Grese T A, Dodge J A, Bryant H U, Turner C H, “Emerging Therapies for the Prevention or Treatment of Post-menopausal Osteoporosis” Journal Of Medicinal Chemistry, (1999), 42, 1-24).

The most accepted method for the treatment of post-menopausal osteoporosis is estrogen replacement therapy (ERT) or hormone replacement therapy (HRT) (Manson J E, Martin K A, “Post-menopausal Hormone Replacement Therapy”, The New England Journal of Medicine, (2001), 345, 34-40). Effect of Hormone replacement therapy i.e., a combination of estrogen and progestin on increasing the bone mineral density is clearly evident. Additionally, observational studies strongly suggest that estrogens protect post-menopausal women from ischemic heart disease, improve HDL cholesterol levels and fibrinogen levels and lower the risk of death due to coronary heart disease. However, while the therapy is generally successful, patient compliance is low, primarily because estrogen treatment frequently produces undesirable side effects, the most dreaded being the development and growth of cancer of the breast and the uterus. Therefore, the risk of hormone replacement therapy may outweigh the benefits after 5-10 years of treatment. As a result, the need for estrogen/compounds which can maintain positive effects on bone and the cardiovascular system, while minimising proliferative effects in the uterus and breast are highly desirable/evident.

Estrogen has long been classified as the female sex hormone and estrogen action is required for the development and function of the female reproductive system. Estrogens exert their biological properties/effects by binding to intracellular receptors forming a complex. After the receptor and bound ligand are transported to the nucleus of the cell, the complex exerts its effect by binding to certain recognition sites on DNA called the estrogen responsive elements (EREs) and allowing certain genes to be expressed. The implantation of fertilised egg in mammals is dependent on a sequential action of estrogen and progesterone on the uterus. Hormone antagonists or antiestrogens inhibit the action of endogenous hormones at the receptor level resulting in inhibition of implantation and this is one of the promising approaches for control of fertility in humans and other animals. Previous studies have revealed that administration of estrogen antagonists or Selective Estrogen Receptor Modulators to mated females prevent implantation. Studies also reveal their action via inhibiting endometrial receptivity to embryonic signal(s) for decidualisation without affecting pre-implantation development of embryos up to the blastocyst stage (Singh M M, “Centchroman, a selective estrogen receptor modulator, as contraceptive and in the management of hormone related clinical disorders”, Medicinal Research Reviews, (2001) 21; 302-347).

Additionally, research in recent years has shown that estrogens have many other homeostatic functions and it plays supportive role in at least three major systems, the cardiovascular system, central nervous system in cognitive and neuronal function and skeletal system in maintaining bone mineral density. Cardiovascular disease is one of the major leading causes of mortality and morbidity in older women. Menopause and aging increase risk of atherosclerosis and coronary artery disease. An altered lipid profile is thought to be associated with this increased risk. The nature of estrogen's ability to regulate serum lipids is not well understood, but evidences indicate that estrogen can up-regulate LDL receptors in liver which act to remove excess cholesterol. Estrogen is believed to directly influence vessel wall compliance, increase vasodilation, reduce peripheral resistance, inhibit the response of blood vessels to injury and prevent development of atherosclerosis (Mendelson M E, Karas R H, “The Protective Effects of Estrogen on the Cardiovascular System” The New England Journal of Medicine, (1999), 340, 1801-1808). Estrogen replacement therapy confers beneficial effects on cardiovascular health of older women and it is reported that they have the return of serum lipid levels to concentrations to those of the pre-menopausal state (Gruber C J, Tschugguel W, Schneeberger C, Huber J C, “Production and Actions of Estrogens” the New England Journal of Medicine, (2002), 346; 340-352). Further, ovarian steroids, particularly estrogens, are of prime importance in the normal maintenance of brain function. The loss of these steroids at menopause, at least in part, accounts for the cognitive decline and neurodegeneration associated with Alzheimer's disease (Bhel C, Skutella T, Lezoualch F, Post A, Widmann M, Newton J, Holsboer F,” “Neuroprotection against Oxidative Stress by Estrogens” Molecular Pharmacology, (1997), 51, 535-541). On the other hand, the highly undesirable side effects of estrogen stimulation for the development and growth of some cancers of breast, uterus and cervix are not acceptable thus limiting its use. From the foregoing discussion, the status of therapies, which may possess the ideal pharmaceutical profile and mimic the actions estrogens on the bone, cardiovascular system and central nervous system without undesirable side effects on uterus and breast, is essentially evident. Therefore, to obtain estrogen pharmaceuticals that will provide optimal health benefit for each intended medical use, it would be best to have compounds that act positively in those tissues where estrogenic stimulation is needed, but are inactive or block estrogen action in those tissues where stimulation poses a risk. Such compounds have been termed as “Selective Estrogen Receptor Modulators” (SERMs). Selective Estrogen Receptor Modulators produce a spectrum of agonistic and antagonistic actions at different target tissues. Certain non-steroidal antiestrogens have been shown to maintain bone mass in ovariectomised rats while antagonise the proliferative effects in the uterus and breast. Tamoxifen and raloxifene both maintain bone mineral density in post-menopausal women and raloxifene prevents fractures of the spine. The selective actions of tamoxifen and raloxifene have successfully been exploited by the pharmaceutical industry. The complementary and interdependent facets of signal transduction pathway, controlled by the structure activity relationship of the estrogen receptor (ER) ligands which can selectively target different body tissues are being used to identify a range of novel targets for new drug discovery programme. Therefore, in recognition of beneficial effects SERMs on a number of body systems, there is a continuing need for the development of potent Selective Estrogen Receptor Modulators that can selectively target different body tissues. Accordingly, the molecules of formula I, their pharmaceutically acceptable salts and compositions thereof and covered under the present invention are proposed to belong to this category of SERMs.

### SUMMARY OF THE INVENTION

In accordance with the principal embodiment, the main objective of the present invention is to provide a novel class of N-substituted dihydrobenzothiepino, dihydrobenoxepino and tetrahydro benzocyclohepta indoles useful as Selective Estrogen Receptor Modulators. Another embodiment of the present invention is to provide novel substituted dihydrobenzothiepino, dihydrobenoxepino and tetrahydro benzocyclohepta indoles of formula I, their pharmaceutically acceptable salts or pharmaceutically acceptable compositions thereof wherein, 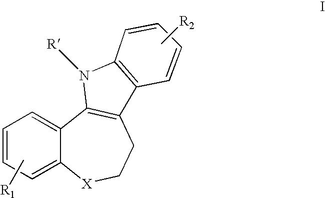
X is —O—, —S— or CH₂,
R′ is Y—(CH₂)ₙ or Y—(CH₂)ₙ—O-Ph
R₁ and R₂ are independently H, OH, —O(C₁-C₆ alkyl), —OCOC₆H₅, —OCO(C₁-C₆ alkyl), —OSO₂(C₄-C₆ alkyl), —OSO₂CF₃, Cl or F;
n is 2 through 6;
Y is selected from the moiety —NR₃R₄ wherein R₃ and R₄ are independently selected from groups consisting such as pyrrolidinoethyl, piperidinoethyl, dimethylaminoethyl, diethylaminoethyl, C₃-C₇ cycloalkyl;
a five membered saturated, unsaturated or partially unsaturated heterocycle containing up to two heteroatoms selected from the groups consisting of —O—, —NH—, —N(C₁C₄ alkyl)-, N═ and —S(O)ₘ, wherein m is an integer of from 0-2, optionally substituted with 1-3 substituents independently selected from the group consisting of H, OH, halo, nitro, cyano, SH, SO₂R₁, CO₂H, CONHR₁, NH₂;
a six membered saturated, unsaturated or partially unsaturated heterocycle containing up to two heteroatoms selected from the groups consisting of —O—, —NH—, —N(C₁C₄ alkyl)-, N═ and —S(O)ₘ, wherein m is an integer of from 0-2, optionally substituted with 1-3 substituents independently selected from the group consisting of H, OH, halo, nitro, cyano, SH, SO₂R₁, CO₂H, CONHR₁, NH₂;
a seven membered saturated, unsaturated or partially unsaturated heterocycle containing up to two heteroatoms selected from the groups consisting of —O—, —NH—, —N(C₁C₄ alkyl)-, N═ and —S(O)ₘ, wherein m is an integer of from 0-2, optionally substituted with 1-3 substituents independently selected from the group consisting of H, OH, halo, nitro, cyano, SH, SO₂R₁, CO₂H, CONHR₁, NH₂; a bicyclic heterocyclic containing from 6-12 carbon atoms either bridged or fused and containing up to two heteroatoms selected from the groups consisting of —O—, —NH—, —N(C₁C₄ alkyl)-, N═ and —S(O)ₘ, wherein m is an integer of from 0-2, optionally substituted with 1-3 substituents independently selected from the group consisting of H, OH, halo, nitro, cyano, SH, SO₂R₁, CO₂H, CONHR₁, NH₂.
   Within the scope of the compounds described above are the preferred compounds of formula I wherein R₁, R₂, R₃, R₄, X, n are as defined above and Y is selected from saturated five, six, seven membered heterocyclic or bicyclic structures with the heteroatoms and substituents described as above.

More particularly, compound of general formula I is useful for the treatment of pathologies mainly associated with post-menopausal syndrome preferably in prevention or treatment of diseases and syndromes caused by:
(a) estrogen deficient or deprivation state in a mammal, in particular osteoporosis, bone loss, bone formation, cardiovascular effects more particularly hyperlipidaemia, thrombosis, vasomotor system and aortal smooth muscle cell proliferation, neurodegenerative effects such as stroke, senile dementia-Alzheimer type and Parkinson disease, menopausal symptoms including hot flushes, urogenital atrophy, depression, mania, schizophrenia and the like, urinary incontinence, relief of dysmenorrhea; relief of dysfunctional uterine bleeding, endometriosis, aid in ovarian development, treatment of acne and hirsutism;
(b) estrogen dependent or estrogen independent cancers such as prostatic carcinoma, cancer of breast, cancer of uterus, cancer of the cervix and cancer of the colon;
(c) an aid in ovarian development or function;
(d) control or regulation of fertility in humans and in other animals;
(e) prevention of threatened or habitual abortion;
(f) suppression of post-partum lactation;
(g) physiological disorders such as obesity, depression etc.; regulation of glucose metabolism in non-insulin dependent diabetes mellitus

### DETAILED DESCRIPTION OF THE INVENTION

The present invention provides a novel class of N— substituted dihydrobenzothiepino, dihydrobenoxepino and tetrahydro benzocyclohepta indoles of formula I, their pharmaceutically acceptable salts or pharmaceutically acceptable compositions thereof wherein, 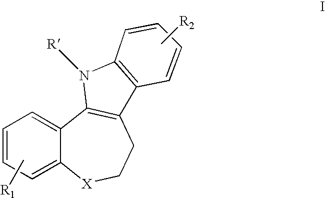
X is —O—, —S— or CH₂,
R′ is Y—(CH₂)ₙ or Y—(CH₂)ₙ—O-Ph
R₁ and R₂ are independently H, OH, —O(C₁-C₆ alkyl), C₁-C₆ alkyloxy-OCOC₆, H5, —OCO(C₁-C₆ alkyl), —OSO₂(C₄-C₆ alkyl), —OSO₂CF₃, Cl or F; n is 2 through 6;
Y is selected from the moiety —NR₃R₄ wherein R₃ and R₄ are independently selected from groups consisting such as pyrrolidinoethyl, piperidinoethyl, dimethylaminoethyl, diethylaminoethyl, C₃-C₇ cycloalkyl;
a five membered saturated, unsaturated or partially unsaturated heterocycle containing up to two heteroatoms selected from the groups consisting of —O—, —NH—, —N(C₁C₄ alkyl)-, N═ and —S(O)ₘ, wherein m is an integer of from 0-2, optionally substituted with 1-3 substituents independently selected from the group consisting of H, OH, halo, nitro, cyano, SH, SO₂R₁, CO₂H, CONHR₁, NH₂;
a six membered saturated, unsaturated or partially unsaturated heterocycle containing up to two heteroatoms selected from the groups consisting of —O—, —NH—, —N(C₁C₄ alkyl, N═ and —S(O)ₘ, wherein m is an integer of from 0-2, optionally substituted with 1-3 substituents independently selected from the group consisting of H, OH, halo, nitro, cyano, SH, SO₂R₁, CO₂H, CONHR₁, NH₂;
a seven membered saturated, unsaturated or partially unsaturated heterocycle containing up to two heteroatoms selected from the groups consisting of —O—, —NH—, —N(C₁C₄ alkyl)-, N═ and —S(O)ₘ, wherein m is an integer of from 0-2, optionally substituted with 1-3 substituents independently selected from the group consisting of H, OH, halo, nitro, cyano, SH, SO₂R₁, CO₂H, CONHR₁, NH₂; a bicyclic heterocyclic containing from 6-12 carbon atoms either bridged or fused and containing up to two heteroatoms selected from the groups consisting of —O—, —NH—, —N(C₁C₄ alkyl)-, N═ and —S(O)ₘ, wherein m is an integer of from 0-2, optionally substituted with 1-3 substituents independently selected from the group consisting of H, OH, halo, nitro, cyano, SH, SO₂R₁, CO₂H, CONHR₁, NH₂.
   General terms used in the description of compounds herein described bear their usual meanings. For example, the term C₁-C₆ alkyl represents straight or branched chain hydrocarbons of 1 to 6 carbon atoms including methyl, ethyl, propyl, butyl, n-butyl, pentyl, hexyl, isopropyl, isobutyl, isopentyl, isohexyl and the like. Similarly the term C₁-C₆ alkyloxy represents alkyl groups as defined above attached to the parent molecular moiety through an oxygen atom and include groups such as methoxy, ethoxy, n-propoxy, isopropoxy, and the like.

The term “pharmaceutically acceptable salts” as used through this specification and the appended claims denotes salts of the types disclosed in the article by Berge et al. (J. Pharmaceutical Sciences, (1977) 66(1), 1-19. The invention includes pharmaceutically acceptable salt forms formed from the addition reaction with either inorganic or organic acids. Inorganic acids such as hydrochloric acid, hydrobromic acid, hydroiodic acid, sulfuric acid, phosphoric acid, nitric acid and the like are useful, as well as mono and dicarboxylic organic acids such as acetic acid, propionic acid, citric acid, maleic acid, tartaric acid, ascorbic acid, fumaric acid, oxalic acid, phthalic acid, succinic acid, methanesulfonic acid, toluensulfonic acid, naphthalenesulfonic acid, camphorsulfonic acid, bensenesulfonic acid. It is known that compounds possessing a basic nitrogen can be complexed with many different acids (both protic and non protic) and usually it is preferred to administer a compound of this invention in the form of acid addition salt.

Additionally, this invention includes quaternary ammonium salts of the compounds herein, which can be prepared by reacting the nucleophilic amines of the side chain with a suitably reactive alkylating agent such as an alkyl halide or benzyl halide.

In an important embodiment, the present invention provides a pharmaceutical composition comprising a therapeutically effective amount of compound of formula I or a pharmaceutically acceptable salt thereof, alone or in a combination of an estrogen or a progestin or both and one or more pharmaceutically acceptable carrier or excipients. In another embodiment, the present invention provides a medical method of employing the compounds of the present invention or pharmaceutically acceptable salts and compositions thereof and methods of using such compounds for the prevention or treatment of symptoms of estrogen deficiency or deprivation including estrogen deficient or deprivation states in mammals, in particular osteoporosis, bone loss, bone formation, cardiovascular effects more particularly hyperlipidaemia.

In another embodiment of the medical methods of the present invention, the compounds of the present invention or pharmaceutically acceptable salts and compositions thereof are employed in the prevention or the treatment of estrogen dependent or independent cancer of breast.

In yet another alternative embodiment of the medical methods of the present invention, the compounds of the present invention are employed in the prevention or the treatment of disease conditions or disorders associated with an aberrant physiological response to endogenous estrogen including control or regulation of fertility in humans and in other animals.

Synthesis of Compound of General Formula I

The synthesis of the compounds of formula I with substitution Y—(CH₂)ₙ and Y—(CH₂)ₙ—O—Ph were described in this invention was accomplished by heating a solution of either 3,4-dihydro-2H-benzo[b]oxepin-5-one, 3,4-dihydro-2H-benzo[b]thiepin-5-one or 6,7,8,9-tetrahydro benzocyclohepten-5-one with the appropriately substituted phenyl hydrazine and a protic acid, preferably acetic acid to yield the desired hydrazones which may either be isolated and then cyclised or the crude reaction mixture is subsequently cyclised on further heating with mineral acid, preferably 20% aqueous hydrochloric acid to give indoles which are precipitated as crystalline solids. The respective indoles are then alkylated at nitrogen with dihaloalkanes or 4-(ω-haloalkoxy) benzyl bromides preferably by deprotonation with a suitably strong base eg. NaH in dimethylformamide and then treated with cyclic or acyclic amines in dry DMF and the presence of suitable catalyst preferably tetrabutyl ammonium iodide to accelerate the reaction to give the desired compounds of formula I as depicted in general Scheme 1: 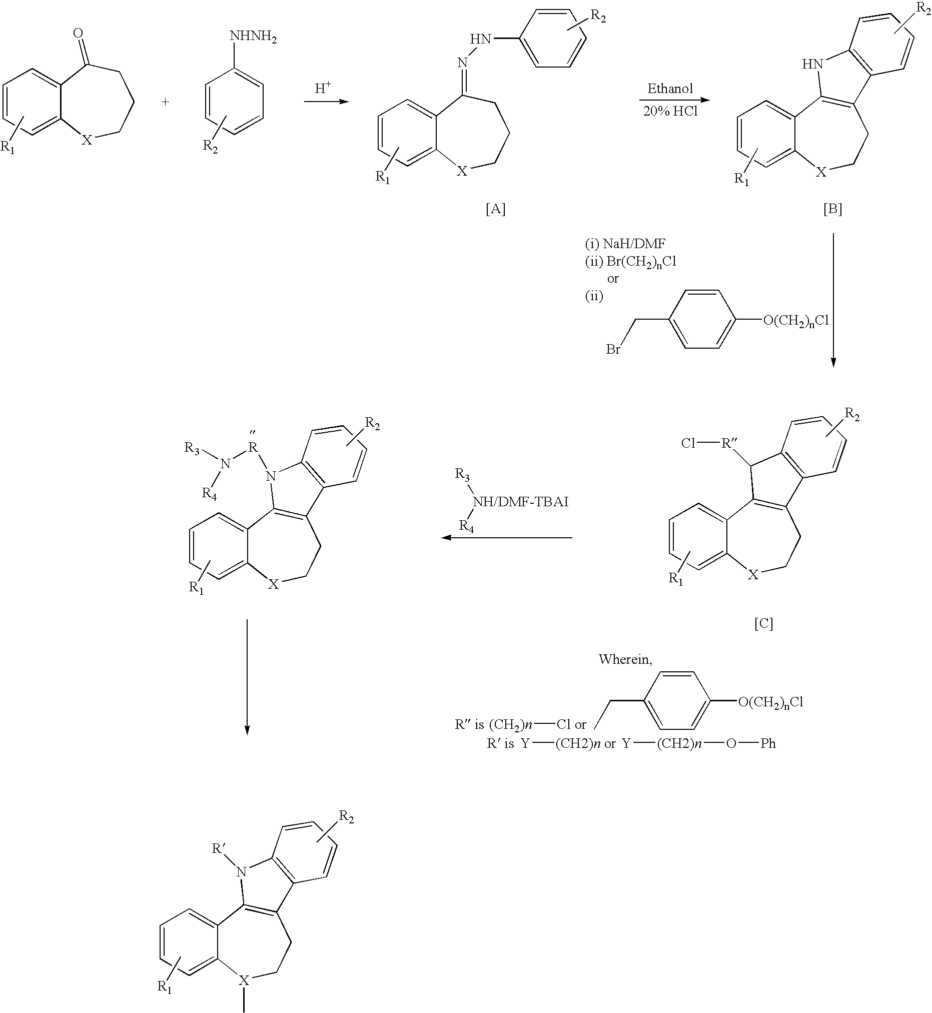
X=S, O, CH₂
n=2-6,
R₁, R₂, R₃, R₄ are independently selected as described
   4-(ω-haloalkoxy) benzyl bromides were synthesized starting with 4-hydroxy benzaldehyde which was alkylated with bromo chloro alkanes to give 4-(ψ-haloalkoxy) benzaldehyde, subsequent reduction of the aldehyde with sodium borohydride in alcohol gave 4-(ω-haloalkoxy) benzyl alcohol. This on reaction with PBr₃ in dioxane gave the desired intermediates of 4-(ω-haloalkoxy) benzyl bromides, as shown in Scheme 2: 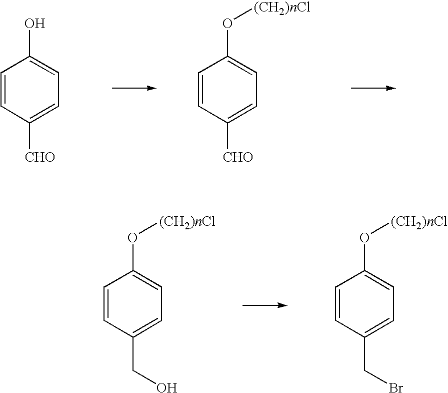
   n is as described

This concept is further illustrated in Scheme 3 for the specific synthesis of compound exemplified as example no. 13 and its ascorbic acid salt as example no. 15: 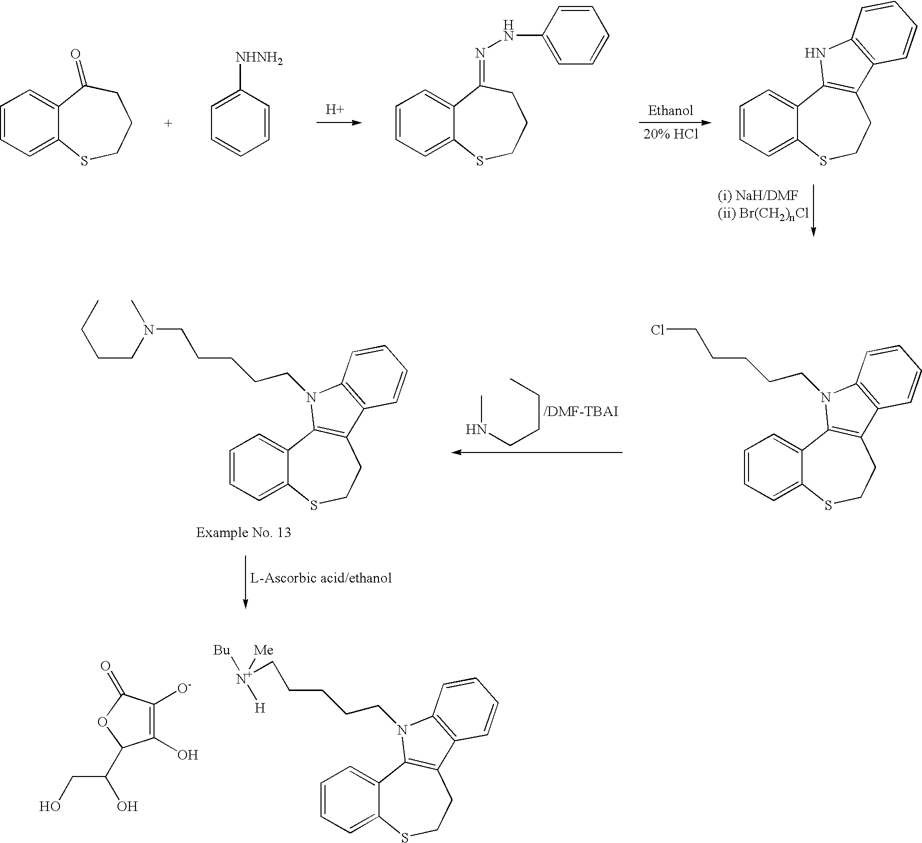
The dihydroxy analogue of compound 13 was synthesized as shown in Scheme 3.

A solution of 8-methoxy-3,4-dihydro-2H-benzo[b]thiepin-5-one and 4-methoxy phenyl hydrazine hydrochloride in alcohol was refluxed for 5 hr to give benzothiepine-5-one phenyl hydrazone. It was then cyclised in the presence of 20% aqueous HCl to give 3,9-dimethoxy benzothiepine[5,4-b]indole. Alkylation at nitrogen with 1-bromo-5-chloro pentane in presence of NaH/DMF formed the 3,9-Dimethoxy-12-[5-chloro pentyl]-6,7-dihydro-12H-benzothiepino [5,4-b]indole, which on reaction with N-methyl butyl amine using DMF and catalytic TBAI to gave the desired intermediate. Finally demethylation with BBr₃/DCM at −5 to 0° C. yielded the compound no. 37 as a solid, as shown in Scheme 4:

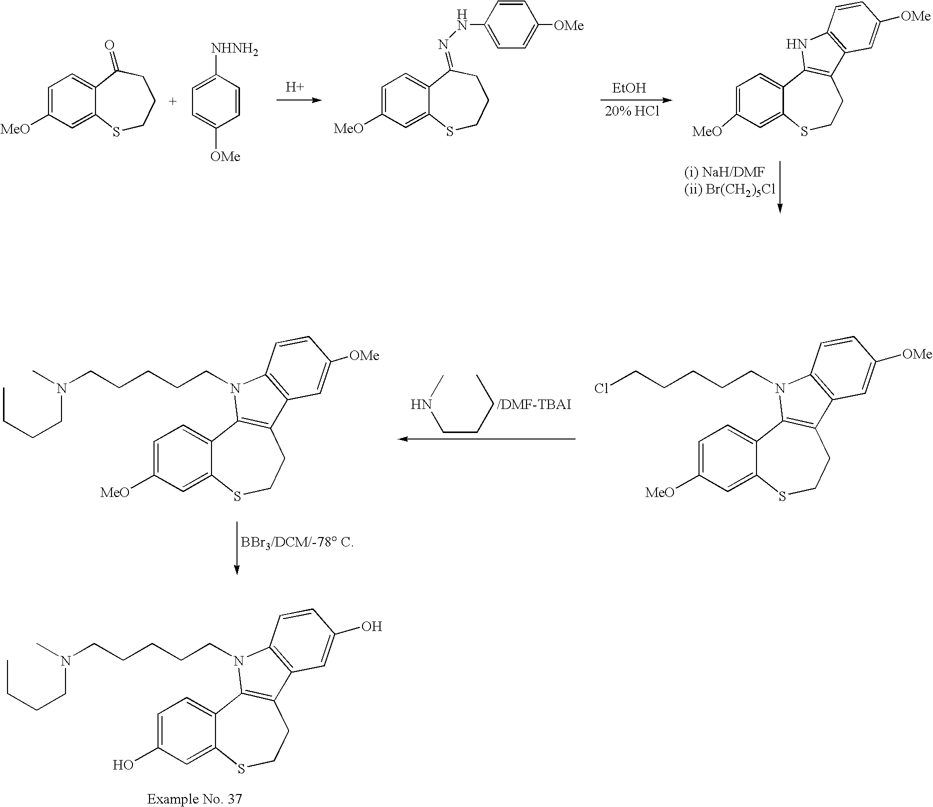

Pharmaceutical compositions of the compound of the present invention or a pharmaceutically acceptable salt thereof may be prepared by procedures known in the art of literature using pharmaceutically acceptable excipients known in the art.

Methods of preventing or treating disorders or disease conditions mentioned herein comprise administering to an individual human being or any other mammal or any other animal in need of such treatment a therapeutically effective amount of one or more of the compounds of this invention or a pharmaceutically acceptable salt or a pharmaceutically acceptable composition thereof with one or more of the pharmaceutically acceptable carriers, excipients etc.

The dosage regimen and the mode of administration of the compound of this invention or a pharmaceutically acceptable salt or a pharmaceutically acceptable composition thereof with one or more of the pharmaceutically acceptable carriers, excipients etc. will vary according to the type of disorder or disease conditions described herein and will be subject to the judgment of the medical practitioner involved.

The compound of this invention or a pharmaceutically acceptable salt or a pharmaceutically acceptable composition with one or more of the pharmaceutically acceptable carriers, excipients etc. may effectively be administered in doses ranging from 0.1 mg to 1000 mg, more preferably in doses ranging from 0.5 mg to 500 mg or still more preferably in the doses ranging from 1 mg to 100 mg bi-weekly or daily or twice a day or three times a day or in still more divided doses.

Therapeutically effective amounts of a compound of the present invention or a pharmaceutically acceptable salt thereof may be enclosed in gelatin capsules or compressed into the tablets or pills or may be formulated in the form of lozenges, inclusion complexes with cyclodextrin derivatives, injectable depo formulations, aerosols, granules, powders, oral liquids, mucosal adhesive formulations, gel formulations, troches, elixirs, suspensions, syrups, wafers, liposomal delivery system, implants, suppository, pessary, microemulsions, nanoemulsion, microparticles, nanoparticles, controlled release delivery system, transdermal delivery systems, targeted delivery systems such as conjugates with monoclonal antibodies or with other suitable carriers moieties.

Such doses may be administered by any appropriate route for example, oral, systemic, local or tropical delivery for example intravenous, intra-arterial, intramuscular, subcutaneous, intraperitonial, intradermal, buccal, intranasal, inhalation, vaginal, rectal, transdermal or any other suitable means in any conventional liquid or solid dosage form to achieve, conventional delivery, controlled delivery or targeted delivery of the compound of this invention or a pharmaceutically acceptable salt or a pharmaceutically acceptable compositions thereof with one or more of the pharmaceutically acceptable carriers, excipients etc.

A preferred mode of administration of a compound of the present invention or a pharmaceutically acceptable salt or a pharmaceutically acceptable composition thereof is oral.

Oral composition will generally compromise of the present invention or a pharmaceutically acceptable salt thereof and one or more of the pharmaceutically acceptable excipients.

The oral composition can be such as tablets; pills, capsules, powders, granules and they may contain any of the following pharmaceutically acceptable excipients:
1. a diluent such as lactose, mannitol, sorbitol, microcrystalline cellulose, sucrose, sodium citrate, dicalcium phosphate or any other ingredient of the similar nature alone in a suitable combination thereof;
2. a binder such as gum tragacanth, gum acacia, methyl cellulose, gelatin, polyvinyl pyrrolidone, starch or any other ingredient of the similar nature alone in a suitable combination thereof;
3. a disintegrating agent such as agar-agar, calcium carbonate, sodium carbonate, silicates, alginic acid, corn starch, potato tapioca starch, primogel or any other ingredient of the similar nature alone or in a suitable combination thereof;
4. a lubricant such as magnesium stearate, talc, solid polyethylene glycols, sodium lauryl sulphate or any other ingredient of similar nature alone or in a suitable combination thereof;
5. a glidant such as colloidal silicon dioxide or any other ingredient of similar nature alone or in a suitable combination thereof;
6. a sweetening agent such as sucrose, saccharin or any other ingredient of similar nature alone or in a suitable combination thereof;
7. a flavoring agent such as peppermint, methyl salicylate, orange flavour, vanilla flavour, or any other pharmaceutically acceptable flavor alone or in a suitable combination thereof;
8. wetting agent such as cetyl alcohol, glyceryl monostearate or any other pharmaceutically acceptable flavor alone or in a suitable combination thereof;
9. absorbents such as kaolin, bentonite clay or any other pharmaceutically acceptable flavor alone or in a suitable combination thereof;
10. Solution retarding agents such as wax, paraffin or any other pharmaceutically acceptable flavor alone or in a suitable combination thereof.

More particularly present invention define a compound of formulas I and pharmaceutically acceptable salt thereof wherein, 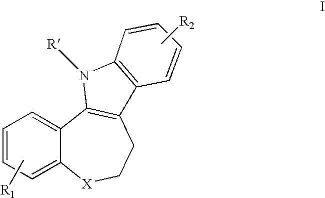
X is —O—, —S— or CH₂,
R′ is Y—(CH₂)ₙ or Y—(CH₂)ₙ—O-Ph
R₁ and R₂ are independently H, OH, —O(C₁-C₆ alkyl), —OCOC₆, H5, —OCO(C₁-C₆ alkyl), OSO₂(C₄C₆alkyl), —OSO₂CF₃, Cl or F; n is 2 through 6; Y is selected from the moiety —NR₃R₄ wherein R₃ and R₄ are independently selected from groups consisting such as pyrrolidinoethyl, piperidinoethyl, dimethylaminoethyl diethylaminoethyl, C₃-C₇ cycloalkyl;
a five membered saturated, unsaturated or partially unsaturated heterocycle containing up to two heteroatoms selected from the groups consisting of —O—, —NH—, —N(C₁C₄ alkyl)-, N═ and —S(O)ₘ, wherein m is an integer of from 0-2, optionally substituted with 1-3 substituents independently selected from the group consisting of H, OH, halo, nitro, cyano, SH, SO₂R, CO₂H, CONHR₁, NH₂;
a six membered saturated, unsaturated or partially unsaturated heterocycle containing up to two heteroatoms selected from the groups consisting of —O—, —NH—, —N(C₁C₄ alkyl, N═ and —S(O)ₘ, wherein m is an integer of from 0-2, optionally substituted with 1-3 substituents independently selected from the group consisting of H, OH, halo, nitro, cyano, SH, SO₂R₁, CO₂H, CONHR₁, NH₂;
a seven membered saturated, unsaturated or partially unsaturated heterocycle containing up to two heteroatoms selected from the groups consisting of —O—, —NH—, —N(C₁C₄ alkyl)-, N═ and —S(O)ₘ, wherein m is an integer of from 0-2, optionally substituted with 1-3 substituents independently selected from the group consisting of H, OH, halo, nitro, cyano, SH, SO₂R₁, CO₂H, CONHR₁, NH₂; a bicyclic heterocyclic containing from 6-12 carbon atoms either bridged or fused and containing up to two heteroatoms selected from the groups consisting of —O—, —NH—, —N(C₁C₄ alkyl)-, N═ and —S(O)ₘ, wherein m is an integer of from 0-2, optionally substituted with 1-3 substituents independently selected from the group consisting of H, OH, halo, nitro, cyano, SH, SO₂R₁, CO₂H, CONHR₁, NH₂.

In another embodiment the compounds of formula I as claimed in claim 1 wherein, R₁ and R₂ are independently selected from H, OH, OCH₃.

Yet another embodiment the compounds of formula I as claimed in claim 1 wherein, n is 5 or 6.

In another embodiment the compounds of formula I as claimed in claim 1 wherein, Y is selected from acyclic or cyclic 5 or 6 membered saturated heterocyclic amine, preferably piperidine, pyrrolidine, N-methylbutylamine and the like.

Yet another embodiment of the present invention is that the compound of formula I as claimed in claim 1 wherein, X is S.

In another embodiment the compound of formula I as claimed in claim 1 wherein, Y is N-methylbutylamine or piperidine.

In another embodiment the compounds as claimed in claim 1, wherein the preferred compound are;
12-[2-(piperidin-1-yl)ethyl]-6,7-dihydro-12H-benzothiepino [5,4-b]indole.
12-[2-(pyrolidin-1-yl ethyl]-6,7-dihydro-12H-benzothiepino [5,4-b]indole.
12-[2-(N-butyl methyl amino)ethyl]-6,7-dihydro-12H-benzothiepino [5,4-b]indole.
12-[3-(piperidin-1-yl)propyl]-6,7-dihydro-12H-benzothiepino [5,4-b]indole.
12-[3-(pyrrolidin-1-yl)propyl]-6,7-dihydro-12H-benzothiepino [5,4-b]indole.
12-[3-(N-butyl methyl amino)propyl]-6,7-dihydro-12H-benzothiepino [5,4-b]indole.
12-[4-(piperidin-1-yl)butyl]-6,7-dihydro-12H-benzothiepino [5,4-b]indole.
12-[4-(pyrrolidin-1-yl)butyl]-6,7-dihydro-12H-benzothiepino [5,4-b]indole.
12-[4-(N-butyl methyl amino)butyl]-6,7-dihydro-12H-benzothiepino [5,4-b]indole.
12-[4-(morpholine-4-yl)butyl]-6,7-dihydro-12H-benzothiepino [5,4-b]indole.
12-[5-(piperidin-1-yl)pentyl]-6,7-dihydro-12H-benzothiepino [5,4-b]indole.
12-[5-(pyrrolidin-1-yl)pentyl]-6,7-dihydro-12H-benzothiepino [5,4-b]indole.
12-[5-(N-butyl methyl amino)pentyl]-6,7-dihydro-12H-benzothiepino [5,4-b]indole.
12-[5-(N-butyl methyl amino pentyl]-6,7-dihydro-12H-benzothiepino [5,4-b]indole methyl iodide salt.
12-[5-(N-butyl methyl amino pentyl]-6,7-dihydro-12H-benzothiepino [5,4-b]indole ascorbic acid salt.
12-[5-(N-butyl methyl amino pentyl]-6,7-dihydro-12H-benzothiepino [5,4-b]indole fumaric acid salt.
12-[5-(morpholin-4-yl)pentyl]-6,7-dihydro-12H-benzothiepino [5,4-b]indole.
9-Methoxy-12-[5-(piperidin-1-yl)pentyl]-6,7-dihydro-12H-benzothiepino[5,4-b]indole.
9-Methoxy-12-[5-(pyrrolidin-1-yl)pentyl]-6,7-dihydro-12H-benzothiepino[5,4-b]indole.
9-Methoxy-12-[5-(N-butyl methyl amino)pentyl]-6,7-dihydro-12H-benzothiepino[5,4-b]indole.
9-Hydroxy-12-[5-(piperidin-1-yl)pentyl]-6,7-dihydro-12H-benzothiepino[5,4-b]indole.
9-Hydroxy-12-[5-(pyrrolidin-1-yl)pentyl]-6,7-dihydro-12H-benzothiepino[5,4-b]indole.
3-Hydroxy-12-[5-(N-butyl methyl amino)pentyl]-6,7-dihydro-12H-benzothiepino[5,4-b]indole.
3-Methoxy-12-[5-(piperidin-1-yl)pentyl]-6,7-dihydro-12H-benzothiepino[5,4-b]indole.
3-Methoxy-12-[5-(pyrrolidin-1-yl)pentyl]-6,7-dihydro-12H-benzothiepino[5,4-b]indole.
3-Methoxy-12-[5-(N-butyl methyl amino)pentyl]-6,7-dihydro-12H-benzothiepino[5,4-b]indole.
3-Hydroxy-12-[5-(piperidin-1-yl)pentyl]-6,7-dihydro-12H-benzothiepino[5,4-b]indole.
3-Hydroxy-12-[5-(pyrrolidin-1-yl)pentyl]-6,7-dihydro-12H-benzothiepino[5,4-b]indole.
3,9-Dimethoxy-12-[5-(piperidin-1-yl)pentyl]-6,7-dihydro-12H-benzothiepino[5,4-b]indole.
3,9-Dimethoxy-12-[5-(pyrrolidin-1-yl)pentyl]-6,7-dihydro-12H-benzothiepino[5,4-b]indole.
3,9-Dimethoxy-12-[5-(N-butyl methyl amino)pentyl]-6,7-dihydro-12H-benzothiepino[5,4-b]indole.
3,9-Dihydroxy-12-[5-(piperidin-1-yl)pentyl]-6,7-dihydro-12H-benzothiepino [5,4-b]indole.
3,9-Dihydroxy-12-[5-(pyrrolidin-1-yl)pentyl]-6,7-dihydro-12H-benzothiepino[5,4-b]indole.
3,9-Dihydroxy-12-[5-(N-butyl methyl amino)pentyl]-6,7-dihydro-12H-benzothiepino[5,4-b]indole.
12-[6-(piperidin-1-yl)hexyl]-6,7-dihydro-12H-benzothiepino[5,4-b]indole.
12-[6-(pyrrolidin-1-yl)hexyl]-6,7-dihydro-12H-benzothiepino[5,4-b]indole.
12-[6-(N-butyl methyl amino)hexyl]-6,7-dihydro-12H-benzothiepino[5,4-b]indole.
12-[6-(morpholin-4-yl)hexyl]-6,7-dihydro-12H-benzothiepino[5,4-b]indole.
12-[4-(2-piperidin-1-yl-ethoxy)benzyl]-6,7-dihydro-12H-benzothiepino[5,4-b]indole.
12-[4-(2-pyrrolodin-1-yl-ethoxy)benzyl]-6,7-dihydro-12H-benzothiepino[5,4-b]indole.
12-[4-(2-(N-butyl methyl amino)-ethoxy)benzyl]-6,7-dihydro-12H-benzothiepino[5,4-b]indole.
12-[4-(3-piperidin-1-yl-propoxy)benzyl]-6,7-dihydro-12H-benzothiepino[5,4-b]indole.
12-[4-(3-pyrrolidin-1-yl-propoxy)benzyl]-6,7-dihydro-12H-benzothiepino[5,4-b]indole
12-[4-{3-(N-butyl methyl amino-propoxy)benzy}]-6,7-dihydro-12H-benzothiepino[5,4-b]indole
12-[4-(5-piperidin-1-yl-pentyloxy)benzyl]-6,7-dihydro-12H-benzothiepino[5,4-b]indole.
12-[4-(5-pyrrolidin-1-yl-pentyloxy)benzyl]-6,7-dihydro-12H-benzothiepino[5,4-b]indole.
12-[4-{5-(N-butylmethylamino)-pentyloxy}benzyl]-6,7-dihydro-12H-benzothiepino[5,4-b]indole.
12-[2-(piperidin-1-yl)ethyl]-6,7-dihydro-12H-benzoxepino[5,4-b]indole.
12-[2-(pyrolidin-1-yl) ethyl]-6,7-dihydro-12H-benzoxepino[5,4-b]indole.
12-[2-(N-butyl methyl amino) ethyl]-6,7-dihydro-12H-benzoxepino[5,4-b]indole.
12-[3-(piperidin-1-yl) propyl]-6,7-dihydro-12H-benzoxepino[5,4-b]indole.
12-[3-(pyrrolidin-1-yl) propyl]-6,7-dihydro-12H-benzoxepino[5,4-b]indole.
12-[3-(N-butyl methyl amino) propyl]-6,7-dihydro-12H-benzoxepino[5,4-b]indole.
12-[5-(piperidin-1-yl) pentyl]-6,7-dihydro-12H-benzoxepino[5,4-b]indole.
12-[5-(pyrrolidin-1-yl) pentyl]-6,7-dihydro-12H-benzoxepino[5,4-b]indole.
12-[5-(N-butyl methyl amino) pentyl]-6,7-dihydro-12H-benzoxepino[5,4-b]indole.
3-Methoxy-12-[5-(piperidin-1-yl)pentyl]-6,7-dihydro-12H-benzoxepino[5,4-b]indole.
3-Methoxy-12-[5-(pyrrolidin-1-yl)pentyl]-6,7-dihydro-12H-benzoxepino[5,4-b]indole.
3-Methoxy-12-[5-(N-butyl methyl amino)pentyl]-6,7-dihydro-12H-benzoxepino[5,4-b]indole.
3,9-Dimethoxy-12-[5-(piperidin-1-yl)pentyl]-6,7-dihydro-12H-benzoxepino[5,4-b]indole.
3,9-Dimethoxy-12-[5-(pyrrolidin-1-yl)pentyl]-6,7-dihydro-12H-benzoxepino[5,4-b]indole.
3,9-Dimethoxy-12-[5-(N-butyl methyl amino)pentyl]-6,7-dihydro-12H-benzoxepino[5,4-b]indole.
12-[4-(2-piperidin-1-yl-ethoxy)benzyl]-6,7-dihydro-12H-benzoxepino[5,4-b]indole.
12-[4-(2-pyrrolodin-1-yl-ethoxy)benzyl]-6,7-dihydro-12H-benzoxepino[5,4-b]indole.
12-[4-{2-(N-butyl methyl amino)-ethoxy}benzyl]-6,7-dihydro-12H-benzoxepino[5,4-b]indole.
12-[4-(3-piperidin-1-yl-propoxy)benzyl]-6,7-dihydro-12H-benzoxepino[5,4-b]indole
12-[4-(3-pyrrolidin-1-yl-propoxy)benzyl]-6,7-dihydro-12H-benzoxepino[5,4-b]indole.
12-[4-{3-(N-butyl methyl amino)-propoxy}benzyl]-6,7-dihydro-12H-benzoxepino[5,4-b]indole.
12-[4-(5-piperidin-1-yl-pentyloxy)benzyl]-6,7-dihydro-12H-benzoxepino[5,4-b]indole.
12-[4-(5-pyrrolidin-1-yl-pentyloxy)benzyl]-6,7-dihydro-12H-benzoxepino[5,4-b]indole.
12-[4-{5-(N-butyl methyl amino)-pentyloxy}benzyl]-6,7-dihydro-12H-benzoxepino[5,4-b]indole.
12-[5-(piperidine-1-yl)-pentyl)]-5,6,7,12-tetrahydro-benzo[6,7]cyclohepta[1,2-b]indole.
12-[5-(pyrrolidine-1-yl)-pentyl)]-5,6,7,12-tetrahydro-benzo[6,7]cyclohepta[1,2-b]indole.
12-[5-(N-butyl methyl amino)-pentyl)]-5,6,7,12-tetrahydro-benzo[6,7]cyclohepta[1,2-b]indole

In further embodiment of the invention is a process for preparation of compound of general formula I as shown in scheme 1 comprises the steps;
(i) reacting a mixture of 3,4-dihydro-2H-benzo[b]thiepin-5-one or 3,4-dihydro-2H-benzo[b]oxepin-5-one or 6,7,8,9-tetrahydro-benzocyclohepten-5-one with substituted hydrazine and a protic acid for 4-5 hrs to form a compound of formula [A] wherein X is S, O, CH₂ and R₁ & R₂ is H.
(ii) reacting a mixture of compound of formula [A] in ethanol and 15-30% aqueous hydrochloric acid for 12-15 hrs to form compound of formula [B], wherein, X is S, O, CH₂, and R₁ and R₂ is H.
(iii) reacting compound [B] with various dihalo compounds in presence of a suitable base in NaH in solvent DMF at 0° C. under stirring conditions to form a compound of formula [C] wherein R″ is (CH₂)ₙ—Cl or -Ph-O—(CH₂)ₙ—Cl X is S, O, CH₂ and n is 2 through 6.
(iv) reacting compound [C] with cyclic or acyclic heteroamine in solvent DMF and under stirring conditions at 70-75° C. in presence of a catalyst to obtain final compounds of formula I, wherein, X is S, O, CH₂, R₁ and R₂ is H and n is 2 through 6. in particular, N-methylbutylamine or piperidine

In another embodiment wherein substituted hydrazine is selected from phenyl hydrazine and 4-methoxy phenyl hydrazine,

In further embodiment wherein in step (i) protic acid is glacial acetic acid.

In another embodiment wherein in step (iii) dihalo compounds are particularly chlorobromoalkane or 4-(ψ-haloalkoxy) benzyl bromides.

In further embodiment in step (iv) cyclic or acyclic heteroamine are selected from N-methylbutyl amine or piperidine.

In another embodiment in step (iv) catalyst is tetrabutyl ammonium iodide.

In yet another embodiment the method of treatment/prevention of estrogen related diseases or syndromes, preferably diseases or syndromes caused by an estrogen-deficient state in humans and in other animals said method comprising administrating pharmaceutical acceptable amount of compound I and its derivatives in a subject.

In an embodiment of the invention, wherein compound I and its derivatives are used for treatment of diseases and syndromes caused by osteoporosis, bone loss, bone fracture, periodontal disease, metastatic bone disease, osteolytic bone disease, post plastic surgery, post-prosthetic joint surgery, and post dental implantation.

In yet another embodiment wherein, said method comprising prevention or treatment is of diseases and syndromes caused by cardiovascular effects more particularly hyperlipidaemia, thrombosis and vasomotor system.

In yet another embodiment wherein, said method comprising prevention or treatment of diseases and syndromes caused by neurodegenerative effects such as stroke, senile dementia-Alzheimer type and Parkinson diseases.

In another embodiment wherein said method comprising prevention or treatment of diseases and syndromes caused by menopausal symptoms including hot flushes, urogenital atrophy, depression, mania, schizophrenia and the like, urinary incontinence, relief of dysmenorrhea; relief of dysfunctional uterine bleeding, an aid in ovarian development, treatment of acne and hirsutism.

Yet another embodiment wherein said method comprising prevention or treatment of estrogen dependent or estrogen independent cancers such as prostatic carcinoma, cancer of breast, cancer of uterus, cancer of the cervix and cancer of the colon.

In another embodiment, wherein said method is an aid in ovarian development or function.

In an embodiment wherein the compound I is used in the control or regulation of fertility in humans and in other animals.

In an embodiment wherein, treatment/prevention of estrogen related diseases is for prevention of threatened or habitual abortion.

Yet another embodiment wherein, said method of treatment if for the suppression of post-partum lactation.

In an embodiment wherein, said method comprising prevention or treatment of physiological disorders such as obesity, depression and related disorders.

One another embodiment wherein, said method comprising the regulation of glucose metabolism in non-insulin dependent diabetes mellitus.

In another embodiment wherein an effective amount of a compound I or a pharmaceutically acceptable salt thereof and a pharmaceutical carrier or diluent or excipients.

In an embodiment wherein therapeutically effective amount of a compound of the present invention may be selected from a dose range of 0.01 mg to 1000 mg.

Yet another embodiment wherein, therapeutically effective amount of a compound of the present invention may preferably be selected from a dose range of 0.5 mg to 500 mg.

In another embodiment wherein, therapeutically effective amount of a compound of the present invention may preferably be selected from a dose range of 1.0 mg to 100 mg.

Yet another embodiment wherein, therapeutically effective amount of a compound of the present invention may be administered as a single dose or in multiple doses.

Following examples are presented to further illustrate the presentation of the compounds of the present invention, which in no way represent a limitation thereof.

### EXAMPLE NO. 1

### 6,7-Dihydro-12H-benzothiepino [5,4-b]indole

A solution of benzothiepine (0.178 g, 1 mM), phenyl hydrazine (0.108 g, 1 mM) and glacial acetic acid (few drops) in ethanol (5 ml) was refluxed on water bath for 5-6 hr. It was then cooled and the solid was filtered. The crude hydrazone was used as such without further purification as it turned coloured on standing at room temp.

A solution of benzothiepine phenyl hydrazone (1 mM) in ethanol (4 ml) and 20% aq. HCl (3 ml) was refluxed for 12 hr. It was poured on ice and neutralized with 5% aq. NaOH. The precipitated solid was filtered and crystallized in methanol to afford an off white solid, 0.20 g, (82%). mp 156° C., ¹H NMR (CDCl₃) δ: 3.27 (t, CH₂), 3.49 (t, CH₂), 7.15-7.25 (m, 4H), 7.37 (t, 1H), 7.57-7.66 (m, 3H), 8.03 (s, 1H). FABMS: m/z 252 (M+1).

### EXAMPLE NO. 2

### 3-Methoxy-6,7-dihydro-12H-benzothiepino [5,4-b]indole

A solution of 8-methoxy benzothiepine (0.208 g, 1 mM), phenyl hydrazine (0.108 g, 1 mM), glacial acetic acid (few drops) in ethanol (5 ml) was refluxed on water bath for 5-6 hr. It was then cooled and the solid was filtered. The crude hydrazone was used as such without further purification as it turned coloured on standing at room temp.

A solution of 8-methoxy benzothiepine phenyl hydrazone in ethanol (4 ml) and 20% aq. HCl (3 ml) was refluxed for 12 hr. It was poured on ice and neutralized with 5% aq. NaOH. The precipitated solid was filtered and crystallized in methanol to afford an off white solid, 0.196 g (70%), mp 128° C. ¹H NMR (CDCl₃) δ: 3.22 (t, CH₂), 3.42 (t, CH₂), 3.87 (s, 3H, OCH₃), 6.91 (dd, 11H), 7.18 (d, 11H), 7.22 (d, 1H), 7.34-7.43 (m, 4H), 7.61 (t, 11H), 7.90 (s, 1H), EIMS: m/z 281 (M+).

### EXAMPLE NO. 3

### 9-Methoxy-6,7-dihydro-12H-benzothiepino [5,4-b]indole

A solution of benzothiepine (0.178 g, 1 mM), 4-methoxy phenyl hydrazine hydrochloride (0.174 g, 1 mM) and glacial acetic acid (few drops) in ethanol (5 ml) was refluxed on water bath for 5-6 hr. cooled, filtered the solid. The crude hydrazone was used as such without further purification as it turned coloured on standing at room temp.

Benzothiepine 4-methoxy phenyl hydrazone was dissolved in ethanol (4 ml) and 20% aq. HCl (3 ml) and the reaction mixture was refluxed for 12 hr. It was poured on ice and neutralized with 5% aq. NaOH. The precipitated solid was filtered and crystallized in methanol to afford an off white solid 0.20 g, (73%), mp 151-154° C. ¹H NMR (CDCl₃) δ: 3.27 (t, CH₂), 3.43 (t, CH₂), 3.84 (s, 3H, OCH₃), 6.91 (dd, 1H), 7.19 (m, 2H), 7.22 (d, 1H), 7.33 (d, 1H), 7.53-7.60 (m, 3H), 7.92 (s, 1H), EIMS: m/z 281 (M+).

### EXAMPLE NO. 4

### 3,9-Dimethoxy-6,7-dihydro-12H-benzothiepino [5,4-b]indole

A solution of 8-methoxy benzothiepine (0.208 g, 1 mM), 4-methoxy phenyl hydrazine hydrochloride (0.174 g, 1 mM) in ethanol (5 ml) was refluxed on water bath for 5-6 hr. cooled, filtered the solid. The crude hydrazone was used as such without further purification as it turned coloured on standing at room temp.

A solution of 8-methoxy benzothiepine 4-methoxy phenyl hydrazone (1 mM) in ethanol (4 ml) and 20% aq. HCl (3 ml) was refluxed for 12 hr. It was poured on ice and neutralized with 5% aq. NaOH. The precipitated solid was filtered and crystallized in methanol to afford an off white solid, 0.21 g (70%), mp 150-151° C., ¹H NMR (CDCl₃) δ: 3.22 (t, CH₂), 3.37 (t, CH₂), 3.83 (s, 3H, OCH₃), 3.87 (s, 3H), 6.85 (m, 2H), 6.98 (d, 1H), 7.15 (d, 1H), 7.25 (dd, 1H), 7.57 (d, 1H), 7.79 (s, 1H), EIMS: m/z 311 (M+).

### EXAMPLE NO. 5

### 12-[2-(Piperidine-1-yl) ethyl]-6,7-dihydro-12H-benzothiepino [5,4-b]indole

To a suspension of NaH (60% suspension in oil, 0.138 g, 5.76 mM) in 5 ml dry DMF was added 6,7-dihydro-12H-benzothiepino [5,4-b]indole (0.360 g, 1.44 mM, dissolved in 5 ml dry DMF) at 0° C. under nitrogen atmosphere with stirring. After 15 min, 1-(2-chloroethyl) piperidine hydrochloride (518 mg, 2.88 mM, dissolved in 15 ml of dry DMF) was added dropwise and continued stirring at room temperature for 3 hr. The reaction mixture was poured into water, extracted with ethyl acetate, dried over sodium sulphate and concentrated. The concentrate was chromatographed on basic alumina using ethyl acetate/hexane (1:5) to yield a white solid, 0.31 g (60%), mp 118° C. ¹H NMR (CDCl₃) δ: 1.39 (m, 2H), 1.50 (m, 4H), 2.30 (m, 4H), 2.52 (t, 2H), 2.99 (bs, 2H), 3.54 (bs, 2H), 4.38 (t, 2H), 7.15-7.32 (m, 2H), 7.40-7.62 (m, 4H), 7.61 (d, J=7.6 Hz, 1H), 7.79 (d, J=7.6 Hz, 1H). EIMS: m/z 362 (M+).

### EXAMPLE NO. 6

### 12-[3-Chloro propyl]-6,7-dihydro-12H-benzothiepino [5,4-b]indole

To a suspension of NaH (60% suspension in oil, 0.072 g, 3 mM) in 5 ml dry DMF was added 6,7-dihydro-12H-benzothiepino [5,4-b]indole (0.5 g, 2 mM, dissolved in 5 ml dry DMF) at 0° C. with stirring under nitrogen atmosphere. After 15 min, 1-bromo-3-chloro propane (0.47 g, 3 mM, dissolved in 10 ml DMF) was added dropwise, and continued stirring at room temperature for 1.5 hr. The reaction mixture was poured into water and extracted with ethyl acetate, dried over sodium sulphate and concentrated. The concentrate was chromatographed on silica gel using ethyl acetate/hexane (1:20) to yield an oil, 0.52 g, (80%). ¹H NMR (CDCl₃) δ: 2.02 (m, 2H), 2.99 (bs, 2H), 3.22 (t, 2H), 3.55 (bs, 2H), 4.49 (t, 2H), 7.15-7.32 (m, 3H), 7.39-7.47 (m, 3H), 7.61 (d, J=7.6 Hz, 1H), 7.79 (d, J=7.6 Hz, 1H). EIMS: m/z 327 (M+).

### EXAMPLE NO. 7

### 12-[3-(Piperidine-1-yl) propyl]-6,7-dihydro-12H-benzothiepino [5,4-b]indole

A solution of 12-(3-chloropropyl)-6,7-dihydro-12H-benzothiepino [5,4-b]indole (0.5 g, 1.5 mM), piperidine (0.5 ml) and tetra butyl ammonium iodide (10 mg) in dry DMF (20 ml) was heated at 70-75° C. with stirring for 7 hr. Reaction mixture was diluted with ethyl acetate, washed with water, dried with sodium sulphate and concentrated. The concentrate was chromatographed on basic alumina using ethyl acetate/hexane (1:50) to yield a white solid, 0.54 g (95%), mp 84-87° C. ¹H NMR (CDCl₃) δ: 1.34 (m, 2H), 1.45 (m, 4H), 1.75 (m, 2H), 2.04 (t, 2H) 2.15 (m, 4H), 2.99 (bs, 2H), 3.54 (bs, 2H), 4.33 (t, 2H), 7.15-7.32 (m, 3H), 7.40-7.62 (m, 3H), 7.61 (d, J=7.6 Hz, 1H), 7.79 (d, J=7.6 Hz, 1H), EIMS: m/z 376 (M+).

### EXAMPLE NO. 8

### 12-[4-Chlorobutyl]-6,7-dihydro-12H-benzothiepino [5,4-b]indole

To a suspension of NaH (60% suspension in oil, 0.144 g, 6 mM) in 5 ml dry DMF was added 6,7-dihydro-12H-benzothiepino [5,4-b]indole (1.04 g, 4 mM, dissolved in 10 ml dry DMF) at 0° C. under nitrogen atmosphere with stirring. After 15 min, 1-bromo-4-chloro butane (1.02 g, 6 mM, dissolved in 15 ml DMF) was added dropwise and continued stirring at room temperature for 3 hr. The reaction mixture was poured into water, extracted with ethyl acetate, dried over sodium sulphate and concentrated. The concentrate was chromatographed on silica gel using ethyl acetate/hexane (1:20) to yield a white solid, 0.54 g (80%), mp 75° C. ¹H NMR (CDCl₃) δ: 1.47 (m, 2H), 1.80 (m, 2H), 2.94 (bs, 2H), 3.28 (t, 2H), 3.55 (bs, 2H), 4.49 (t, 2H), 7.16-7.29 (m, 3H), 7.38-7.46 (m, 3H), 7.63 (d, J=7.6 Hz, 1H), 7.79 (d, J=7.6 Hz, 1H). FABMS: m/z 342 (M+1).

### EXAMPLE NO. 9

### 12-[4-(Piperidine-1-yl) butyl]-6,7-dihydro-12H-benzothiepino [5,4-b]indole

A solution of 12-(4-chlorobutyl)-6,7-dihydro-12H-benzothiepino [5,4-b]indole (0.5 g, 1.5 mM), piperidine (0.5 ml), tetra butyl ammonium iodide (10 mg) in dry DMF (20 ml) was heated at 70-75° C. with stirring for 7 hr. On completion, the reaction mixture was diluted with ethyl acetate, washed with water, dried over sodium sulphate and concentrated. The residue obtained was chromatographed on basic alumina using ethyl acetate/hexane (1:50) to yield an oil, 0.48 g (84%). ¹H NMR (CDCl₃) δ: 1.34 (m, 2H), 1.39 (m, 2H), 1.50 (m, 4H), 1.70 (m, 2H), 2.30 (m, 4H), 2.56 (t, 2H), 2.99 (bs, 2H), 3.54 (bs, 2H), 4.38 (t, 2H), 7.15-7.32 (m, 3H), 7.40-7.62 (m, 3H), 7.61 (d, J=7.6 Hz, 1H), 7.79 (d, J=7.6 Hz, 1H). FABMS: m/z 392 (M+1).

### EXAMPLE NO. 10

### 12-(5-Chloro pentyl)-6,7-dihydro-12H-benzothiepino [5,4-b]indole

To a suspension of NaH (60% suspension in oil, 0.72 g, 30 mM) in 20 ml dry DMF was added 6,7-dihydro-12H-benzothiepino [5,4-b]indole (5.02 g, 20 mM, dissolved in 30 ml dry DMF) at 0° C. under nitrogen atmosphere with stirring. After 15 min, 1-bromo-5-chloro pentane (5.5 g, 30 mM, dissolved in 30 ml DMF) was added dropwise and continued stirring at room temperature for 1.5 hr. The reaction mixture was poured into water, extracted with ethyl acetate and dried over sodium sulphate. The concentrate was chromatographed on silica gel using ethyl acetate/hexane (1:20) to yield an oil, 6.40 g, (90%). ¹H NMR (CDCl₃) δ: 1.29 (m, 2H), 1.63 (m, 4H), 2.99 (bs, 2H), 3.18 (t, 2H), 3.55 (bs, 2H), 4.29 (t, 2H), 7.15-7.25 (m, 3H), 7.38 (m, 3H), 7.62 (d, J=7.6 Hz, 1H), 7.79 (d, J=7.6 Hz, 1H). FABMS: m/Z 356 (M+1).

### EXAMPLE NO. 11

### 12-[5-(Piperidine-1-yl) pentyl]-6,7-dihydro-12H-benzothiepino [5,4-b]indole

A solution of 12-(5-chloropentyl)-6,7-dihydro-12H-benzothiepino [5,4-b]indole (0.5 g, 1.5 mM), piperidine (0.5 ml) and tetra butyl ammonium iodide (10 mg) in dry DMF (20 ml) was heated at 70-75° C. with stirring for 7 hr. On completion, the reaction mixture was diluted with ethyl acetate, washed with water, dried over sodium sulphate and concentrated. The concentrate was chromatographed on basic alumina using ethyl acetate/hexane (1:50) to yield an oil, 0.48 g (92%). ¹H NMR (CDCl₃) δ: 1.25 (m, 2H), 1.29 (m, 2H), 1.35 (m, 4H) 1.53 (m, 4H), 2.07 (t, 2H), 2.25 (m, 4H), 2.99 (bs, 2H), 3.55 (bs, 2H), 4.26 (t, 2H), 7.15-7.32 (m, 3H), 7.41 (m, 3H), 7.62 (d, J=7.6 Hz, 1H), 7.79 (d, J=7.6 Hz, 1H). FABMS: m/z 405 (M+1).

### EXAMPLE NO. 12

### 12-[5-(Pyrrolidine-1-yl) pentyl]-6,7-dihydro-12H-benzothiepino [5,4-b]indole

A solution of 12-(5-chloropentyl)-6,7-dihydro-12H-benzothiepino [5,4-b]indole (0.5 g, 1.5 mM), pyrrolidine (0.5 ml) and tetra butyl ammonium iodide (10 mg) in dry DMF (20 ml) was heated at 70-75° C. with stirring for 7 hr. On completion, the reaction mixture was diluted with ethyl acetate, washed with water, dried over sodium sulphate and concentrated. The concentrate was chromatographed on basic alumina using ethyl acetate/hexane (1:50) to yield an oil, 0.43 g (88.8%). ¹H NMR (CDCl₃) B: 1.25 (m, 2H), 1.28 (m, 4H), 1.64 (m, 4H), 2.23 (t, 2H), 2.32 (m, 4H), 2.99 (bs, 2H), 3.54 (bs, 2H), 4.38 (t, 2H), 7.15-7.32 (m, 3H), 7.45 (m, 3H), 7.62 (d, J=7.6 Hz, 1H), 7.79 (d, J=7.6 Hz, 1H). FABMS: m/z 391 (M+1).

### EXAMPLE NO. 13

### 12-[5-(N-butyl methyl amino) pentyl]-6,7-dihydro-12H-benzothiepino [5,4-b]indole

A solution of 12-(5-chloropentyl)-6,7-dihydro-12H-benzothiepino [5,4-b]indole (0.5 g, 1.5 mM), N-methyl butyl amine (0.5 ml) and tetra butyl ammonium iodide (10 mg) in dry DMF (20 ml) was heated at 70-75° C. with stirring for 7 hr. On completion, the reaction mixture was diluted with ethyl acetate, washed with water, dried over sodium sulphate and concentrated. The concentrate was chromatographed on basic alumina using ethyl acetate/hexane (1:50) to yield an oil, 0.48 g (84.2%). ¹H NMR (CDCl₃) δ: 0.93 (t, 3H), 1.11 (m, 2H), 1.25 (m, 6H), 1.63 (m, 4H), 2.14 (s, 3H), 2.31 (t, 2H), 2.96 (bs, 2H), 3.55 (bs, 2H), 4.28 (t, 2H), 7.19 (m, 2H), 7.22 (m, 1H), 7.45 (m, 3H), 7.62 (d, J=7.6 Hz, 1H), 7.79 (d, J=7.6 Hz, 1H). FABMS: m/z 406 (M+1).

### EXAMPLE NO. 14

### 12-[5-(N-butyl methyl amino) pentyl]-6,7-dihydro-12H-benzothiepino [5,4-b]indole methyl iodide salt

A solution of 12-[5-(N-butyl methyl amino) pentyl]-6,7-dihydro-12H-benzothiepino [5,4-b]indole (2.5 g), methyl iodide (3 ml, in excess) and dry acetone (30 ml) was stirred at room temperature for 30 min. The precipitated solid was filtered. It was crystallized in ethanol to afford white amorphous solid, 2.95 g (87.5%), mp 112-114° C. ¹H NMR (DMSO-D₆) δ: 0.92 (m, 5H), 1.26 (m, 2H), 1.39 (m, 2H), 1.52 (m, 4H), 2.87 (s, 6H, 2×NCH₃), 3.01 (t, 2H), 3.13 (t, 2H), 3.51 (bs, 4H), 4.32 (t, 2H), 7.10 (t, 1H), 7.22 (t, 1H), 7.40 (t, 1H), 7.62 (m, 4H), 7.79 (d, J=7.6 Hz, 1H). FABMS: m/z 421 (M—I).

### EXAMPLE NO. 15

### 12-[5-(N-butyl methyl amino) pentyl]-6,7-dihydro-12H-benzothiepino [5,4-b]indole ascorbic acid salt

A solution of 12-[5-(N-butyl methyl amino) pentyl]-6,7-dihydro-12H-benzothiepino [5,4-b]indole (2.5 g, 6.15 mM), ascorbic acid (1.08 g, 6.15 mM) and absolute ethanol (30 ml) was warmed on water bath for 10 min, alcohol was evaporated and the product was isolated as pale yellow coloured foam. It could not be crystallized and was purified as foam from ethanol, 3.1 g (86.5%), mp 102-104° C. ¹H NMR (CDCl₃) δ: 0.91 (m, 5H), 1.28 (m, 4H), 1.51 (m, 4H), 2.50 (s, 3H. NCH₃), 2.63 (m, 2H), 2.76 (m, 2H), 3.72 (bs, 2H), 4.24 (m, 2H), 4.29 (t, 2H), 4.63 (m, 1H), 7.16 (t, 1H), 7.27 (m, 2H), 7.44 (m, 3H), 7.57 (d, J=6 Hz, 1H), 7.77 (d, J=8.6 Hz, 1H). FABMS: m/z 407 (M−175).

### EXAMPLE NO. 16

### 12-[5-(N-butyl methyl amino) pentyl]-6,7-dihydro-12H-benzothiepino [5,4-b]indole fumaric acid Salt

A solution of 12-[5-(N-butyl methyl amino) pentyl]-6,7-dihydro-12H-benzothiepino [5,4-b]indole (2.5 g, 6.15 mM), fumaric acid (0.71 g, 6.15 mM) and absolute ethanol (40 ml) was warmed on water bath for 10 min. It was cooled and precipitated product was crystallized in ethanol to afford a white crystalline solid, 2.39 g (74.4%), mp 162-164° C. ¹H NMR (CDCl₃) δ: 0.91 (m, 5H), 1.28 (m, 4H), 1.51 (m, 4H), 2.50 (s, 3H, NCH₃), 2.63 (m, 2H), 2.76 (m, 2H), 3.72 (bs, 2H), 4.24 (m, 2H), 4.29 (t, 2H), 4.63 (m, 1H), 7.16 (t, 1H), 7.27 (m, 2H), 7.44 (m, 3H), 7.57 (d, J=6 Hz, 1H), 7.77 (d, J=8.6 Hz, 1H). FABMS: m/z 407 (M−115).

### EXAMPLE NO. 17

### 12-(6-Chloro hexyl)-6,7-dihydro-12H-benzothiepino [5,4-b]indole

To a suspension of NaH (60% suspension in oil) (0.72 g, 30 mM) in 20 ml dry DMF was added 6,7-dihydro-12H-benzothiepino [5,4-b]indole (5.02 g, 20 mM, dissolved in 30 ml dry DMF) at 0° C. under nitrogen atmosphere with stirring. After 15 min, 1-bromo-6-chloro hexane (4.0 g, 24 mM, dissolved in 20 ml DMF) was added dropwise and continued stirring at room temperature for 1.5 hr. On completion, the reaction mixture was poured into water, extracted with ethyl acetate and dried over sodium sulphate. The concentrate was chromatographed on silica gel using ethyl acetate/hexane (1:20) to yield an oil, 6.61 g (90%). ¹H NMR (CDCl₃) δ: 1.07 (m, 2H), 1.64 (m, 6H), 2.99 (bs, 2H), 3.36 (t, 2H), 3.55 (bs, 2H), 4.29 (t, 2H), 7.15 (m, 1H), 7.15 (m, 2H), 7.41 (m, 3H), 7.58 (d, J=7.6 Hz, 1H), 7.79 (d, J=7.6 Hz, 1H). FABMS: m/z 370 (M+1).

### EXAMPLE NO. 18

### 12-[6-(Piperidine-1-yl)hexyl]-6,7-dihydro-12H-benzothiepino [5,4-b]indole

A solution of 12-(6-chloro hexyl)-6,7-dihydro-12H-benzothiepino [5,4-b]indole (0.5 g, 1.5 mM), piperidine (0.5 ml) and tetra butyl ammonium iodide (10 mg) in dry DMF (15 ml) was heated at 70-75° C. with stirring for 7 hr. On completion, the reaction mixture was diluted with ethyl acetate, washed with water, dried over sodium sulphate and concentrated. The concentrate was chromatographed on basic alumina using ethyl acetate/hexane (1:50) to yield an oil, 0.47 g (83%). ¹H NMR (CDCl₃) δ: 1.07 (m, 2H), 1.30 (m, 2H), 1.41 (m, 4H), 1.56 (m, 6H),) 2.23 (t, 2H), 2.37 (m, 4H), 2.99 (bs, 2H), 3.54 (bs, 2H), 4.38 (t, 2H), 7.18 (m, 1H), 7.25 (m, 2H), 7.40-7.62 (m, 3H), 7.61 (d, J=7.6 Hz, 1H), 7.79 (d, J=7.6 Hz, 1H). FABMS: m/z 419 (M+1).

### EXAMPLE NO. 19

### 12-[6-(Pyrrolidine-1-yl)hexyl]-6,7-dihydro-12H-benzothiepino [5,4-b]indole

A solution of 12-(6-chloro hexyl)-6,7-dihydro-12H-benzothiepino [5,4-b]indole. (0.5 g, 1.5 mM), pyrrolidine (0.5 ml) and tetra butyl ammonium iodide (10 mg) in dry DMF (15 ml) was heated at 70-75° C. with stirring for 7 hr. On completion, the reaction mixture was diluted with ethyl acetate, washed with water, dried over sodium sulphate and concentrated. The concentrate was chromatographed on basic alumina using ethyl acetate/hexane (1:50) to yield an oil, 0.45 g (82.2%). ¹H NMR (CDCl₃) 6:1.09 (m, 4H), 1.33 (m, 2H), 1.62 (m, 2H), 1.80 (m, 4H), 2.26 (t, 2H), 2.31 (m, 4H), 2.95 (bs, 2H), 3.55 (bs, 2H), 4.27 (t, 2H), 7.15 (t, 1H), 7.28 (m, 2H), 7.45 (m, 3H), 7.62 (d, J=7.6 Hz, 1H), 7.79 (d, J=7.6 Hz, 1H). FABMS: m/z 405 (M+1).

### EXAMPLE NO. 20

### 12-[6-(N-butyl methyl amino)hexyl]-6,7-dihydro-12H-benzothiepino [5,4-b]indole

A solution of 12-(6-chloro hexyl)-6,7-dihydro-12H-benzothiepino [5,4-b]indole. (0.5 g, 1.5 mM), N-methyl butyl amine (0.5 ml) and tetra butyl ammonium iodide (10 mg) in dry DMF (15 ml) was heated at 70-75° C. with stirring for 7 hr. Reaction mixture diluted with ethyl acetate, washed with water, dried over sodium sulphate and concentrated. The concentrate was chromatographed on basic alumina using ethyl acetate/hexane (1:50) to yield an oil, 0.43 g (75%). ¹H NMR (CDCl₃) δ: 0.93 (t, 3H), 1.11 (m, 4H), 1.25 (m, 6H), 1.63 (m, 2H), 2.23 (t, 2H), 2.37 (m, 4H), 2.99 (bs, 2H), 3.54 (bs, 2H), 4.38 (t, 2H), 7.15-7.32 (m, 3H), 7.40-7.62 (m, 3H), 7.61 (d, 1H, J=7.6 Hz), 7.79 (d, 1H, J=7.6 Hz). FABMS: m/z 421 (M+1).

### EXAMPLE NO. 21

### 3-Methoxy-12-(5-chloro pentyl)-6,7-dihydro-12H-benzothiepino [5,4-b]indole

To a suspension of NaH (60% suspension in oil, 0.36 g, 15 mM) in 10 ml dry DMF was added 3-methoxy-6,7-dihydro-12H-benzothiepino [5,4-b]indole (2.81 g, 10 mM, dissolved in 20 ml dry DMF) at 0° C. under nitrogen atmosphere with stirring. After 15 min, 1-bromo-5-chloro pentane (2.77 g, 15 mM, dissolved in 20 ml DMF) was added dropwise and continued stirring at room temperature for 1.5 hr. On completion, the reaction mixture was poured into water, extracted with ethyl acetate and dried over sodium sulphate. The concentrate was chromatographed on silica gel using ethyl acetate/hexane (1:20) to yield an oil, 3.26 g (85%). ¹H NMR (CDCl₃) δ: 1.25 (m, 2H), 1.57 (m, 4H), 2.99 (bs, 2H), 3.36 (t, 2H), 3.56 (bs, 2H), 3.81 (s, 3H), 4.49 (t, 2H), 6.97 (dd, J=2.66, 8.56 Hz, 1H), 7.19 (m, 1H), 7.25 (dd, 1H), 7.32 (m, 1H), 7.34 (m, 1H), 7.40 (d, J=7.6 Hz, 1H), 7.55 (d, J=7.6 Hz, 1H). EIMS: m/z 385 (M+).

### EXAMPLE NO. 22

### 3-Methoxy-12-[5-(piperidine-1-yl) pentyl]-6,7-dihydro-12H-benzothiepino [5,4-b]indole

A solution of 3-methoxy-12-(5-chloropentyl)-6,7-dihydro-12H-benzothiepino [5,4-b]indole. (1.0 g, 3.5 mM), piperidine (1.0 ml) and tetra butyl ammonium iodide (20 mg) in dry DMF (20 ml) was heated at 70-75° C. with stirring for 7 hr. On completion, the reaction mixture was diluted with ethyl acetate, washed with water, dried over sodium sulphate and concentrated. The concentrate was chromatographed on basic alumina using ethyl acetate/hexane (1:50) to yield an oil, 0.96 g (85.2%). ¹H NMR (CDCl₃) δ: 1.13 (m, 2H), 1.41 (m, 4H), 1.56 (m, 6H), 2.15 (t, 2H), 2.25 (m, 4H), 2.99 (bs, 2H), 3.54 (bs, 2H), 3.87 (s, 3H, OCH₃), 4.38 (t, 2H), 7.01 (dd, J=2.66, 8.56 Hz, 11H), 7.18 (m, 11H), 7.26 (dd, 1H), 7.32 (d, 1H), 7.34 (d, 11H), 7.40 (d, J=7.6 Hz, 1H), 7.55 (d, J=7.6 Hz, 1H). EIMS: m/z 434 (M+).

### EXAMPLE NO. 23

### 3-Methoxy-12-[5-(pyrrolidine-1-yl) pentyl]-6,7-dihydro-12H-benzothiepino [5,4-b]indole

A solution of 3-methoxy-12-(5-chloropentyl)-6,7-dihydro-12H-benzothiepino [5,4-b]indole. (1.0 g, 3.5 mM), pyrrolidine (1.0 ml) and tetra butyl ammonium iodide (20 mg) in dry DMF (20 ml) was heated at 70-75° C. with stirring for 7 hr. On completion, the reaction mixture was diluted with ethyl acetate, washed with water, dried over sodium sulphate and concentrated. The concentrate was chromatographed on basic alumina using ethyl acetate/hexane (1:50) to yield an oil, 0.95 g (87%). ¹H NMR (CDCl₃) δ: 1.17 (m, 2H), 1.35 (m, 2H), 1.68 (m, 2H), 1.74 (m, 4H), 2.26 (t, 2H), 2.36 (m, 4H), 2.99 (bs, 2H), 3.54 (bs, 2H), 3.87 (s, 3H, OCH₃), 4.23 (t, 2H), 7.02 (dd, J=2.66, 8.56 Hz, 1H), 7.18 (m, 1H), 7.26 (dd, 1H), 7.32 (d, 1H), 7.34 (d, 1H), 7.40 (d, J=7.6 Hz, 1H), 7.59 (d, J=7.6 Hz, 1H). EIMS: m/z 434 (M+).

### EXAMPLE NO. 24

### 3-Hydroxy-12-[5-(piperidine-1-yl) pentyl]-6,7-dihydro-12H-benzothiepino [5,4-b]indole

To a solution of 3-methoxy-12-[5-(piperidine-1-yl) pentyl]-6,7-dihydro-12H-benzothiepino [5,4-b]indole (0.5 g, 1.15 mM) in dry DCM (20 ml), BBr₃ (1M soln in DCM, 5.76 ml, 5.56 mM) was added dropwise under nitrogen atmosphere at −10° C. The reaction mixture was stirred for 3 hr. then allowed to attain room temp. Reaction mixture was quenched with methanol, saturated with aq. NaHCO₃ soln, extracted with DCM, dried over sodium sulphate and concentrated. The concentrate was chromatographed over silica gel in methanol chloroform (1:20) to afford a white solid, 0.36 g (75%). mp 130° C. ¹H NMR (DMSO-D₆) δ: 0.99 (m, 2H), 1.38 (m, 4H), 1.47 (m, 2H), 1.63 (m, 2H), 1.72 (m, 2H), 2.73 (m, 6H), 3.28 (bs, 2H), 3.46 (bs, 2H), 4.28 (t, 2H), 6.96 (dd, 1H, J=8.4, 2.4 Hz), 7.09 (t, 1H), 7.19 (m, 2H), 7.40 (d, J=8.4 Hz, 1H), 7.54 (d, J=8.1 Hz, 1H), 7.59 (d, J=8.1 Hz, 1H). FABMS: m/z 421 (M+1).

### EXAMPLE NO. 25

### 3-Hydroxy-12-[5-(pyrrolidine-1-yl) pentyl]-6,7-dihydro-12H-benzothiepino [5,4-b]indole

To a solution of 3-methoxy-12-[5-(pyrrolidine-1-yl) pentyl]-6,7-dihydro-12H-benzothiepino [5,4-b]indole (0.5 g, 1.18 mM) in dry DCM (20 ml), BBr₃ (1M soln in DCM, 5.76 ml, 5.56 mM) was added dropwise under nitrogen atmosphere at −10° C. The reaction mixture was stirred for 3 hr. then allowed to attain room temp. Reaction mixture was quenched with methanol, saturated with aq. NaHCO₃ soln, extracted with DCM, dried over sodium sulphate and concentrated. The concentrate was chromatographed over silica gel in methanol chloroform (1:20) to afford a white solid, 0.33 g (68%). mp 158° C. ¹H NMR (DMSO-D₆) δ: 1.0 (m, 2H), 1.46 (m, 4H), 1.86 (m, 4H), 2.84 (m, 4H), 3.28 (bs, 2H), 3.55 (bs, 2H), 4.24 (t, 2H), 6.97 (dd, J=8.4, 2.4 Hz, 1H), 7.08 (t, 1H), 7.19 (m, 2H), 7.39 (d, J=8.4 Hz, 1H), 7.54 (d, J=7.8 Hz, 1H), 7.59 (d, J=7.8 Hz, 1H). FABMS: m/z 421 (M+1).

### EXAMPLE NO. 26

### 9-Methoxy-12-(5-chloro pentyl)-6,7-dihydro-12H-benzothiepino [5,4-b]indole

To a suspension of NaH (60% suspension in oil, 0.36 g, 15 mM) in 10 ml dry DMF was added 9-methoxy-6,7-dihydro-12H-benzothiepino [5,4-b]indole (2.81 g, 10 mM, dissolved in 20 ml dry DMF) at 0° C. under nitrogen atmosphere with stirring. After 15 min, 1-bromo-5-chloro pentane (2.77 g, 15 mM, dissolved in 20 ml DMF) was added dropwise and continued stirring at room temperature for 1.5 hr. The reaction mixture was poured into water, extracted with ethyl acetate and dried over sodium sulphate. The concentrate was chromatographed on silica gel using ethyl acetate/hexane (1:20) to yield an oil, 3.46 g (90%). ¹H NMR (CDCl₃) δ: 1.16 (m, 2H), 1.63 (m, 4H), 2.99 (bs, 2H), 3.35 (t, 2H), 3.54 (bs, 2H), 3.88 (s, 3H, OCH₃), 4.25 (t, 2H), 6.90 (dd, J=2.36, 8.8 Hz, 1H), 7.04 (d, J=2.26 Hz, 1H), 7.27 (m, 2H), 7.39 (m, 2H), 7.78 (d, J=7.56 Hz, 1H). EIMS: m/z 385 (M+).

### EXAMPLE NO. 27

### 9-Methoxy-12-[5-(piperidine-1-yl) pentyl]-6,7-dihydro-12H-benzothiepino [5,4-b]indole

A solution of 9-methoxy-12-(5-chloropentyl)-6,7-dihydro-12H-benzothiepino [5,4-b]indole (1.0 g, 3.5 mM), piperidine (1.0 ml), tetra butyl ammonium iodide (20 mg) in dry DMF (20 ml) was heated at 70-75° C. with stirring for 7 hr. On completion, the reaction mixture was diluted with ethyl acetate, washed with water, dried with sodium sulphate and concentrated. The concentrate was chromatographed on basic alumina using ethyl acetate/hexane (1:50) to yield an oil, 1.04 g (92%). ¹H NMR (CDCl₃) δ: 1.10 (m, 2H), 1.34 (m, 6H), 1.61 (m, 4H),) 2.15 (t, 2H), 2.26 (m, 4H), 2.99 (bs, 2H), 3.54 (bs, 2H), 3.88 (s, 3H, OCH₃), 4.22 (t, 2H), 6.90 (dd, J=2.36, 8.8 Hz, 1H), 7.04 (d, J=2.26 Hz, 1H), 7.27 (m, 2H), 7.39 (m, 2H), 7.78 (d, J=7.56 Hz, 1H). EIMS: m/z 438 (M+).

### EXAMPLE NO. 28

### 9-Methoxy-12-[5-(pyrrolidin-1-yl) pentyl]-6,7-dihydro-12H-benzothiepino [5,4-b]indole

A solution of 9-methoxy-12-(5-chloropentyl)-6,7-dihydro-12H-benzothiepino [5,4-b]indole (1.0 g, 3.5 mM), pyrrolidine (1.0 ml), tetra butyl ammonium iodide (20 mg) in dry DMF (20 ml) was heated at 70-75° C. with stirring for 7 hr. On completion, the reaction mixture was diluted with ethyl acetate, washed with water, dried with sodium sulphate and concentrated. The concentrate was chromatographed on basic alumina using ethyl acetate/hexane (1:50) to yield an oil, 0.92 g (85%). ¹H NMR (CDCl₃) δ: 1.14 (m, 2H), 1.33 (m, 2H), 1.69 (m, 2H), 1.75 (m, 4H), 2.21 (t, 2H), 2.36 (m, 4H), 2.99 (bs, 2H), 3.54 (bs, 2H), 3.88 (s, 3H, OCH₃), 4.22 (t, 2H), 6.93 (dd, J=2.36, 8.8 Hz, 1H), 7.04 (d, J=2.26 Hz, 1H), 7.31 (m, 2H), 7.43 (m, 2H), 7.78 (d, J=7.56 Hz, 1H). EIMS: m/z 421 (M+).

### EXAMPLE NO. 29

### 9-Hydroxy-12-[5-(piperidine-1-yl) pentyl]-6,7-dihydro-12H-benzothiepino [5,4-b]indole

To a solution of 9-methoxy-12-[5-(piperidine-1-yl) pentyl]-6,7-dihydro-12H-benzothiepino [5,4-b]indole (0.5 g, 1.15 mM) in dry DCM (20 ml), BBr₃ (1M soln in DCM, 5.76 ml, 5.56 mM) was added dropwise under nitrogen atmosphere at −10° C. The reaction mixture was stirred for 3 hr. then allowed to attain room temp. The reaction mixture was quenched with methanol and saturated with aq. NaHCO₃ soln. It was extracted with DCM, dried over sodium sulphate and concentrated. The concentrate was chromatographed over silica gel in methanol chloroform (1:20) to afford a white solid, 0.29 g (60%), mp 182° C. ¹H NMR (DMSO-D₆) δ: 1.07 (m, 2H), 1.38 (m, 4H), 1.59 (m, 6H), 2.12 (t, 2H), 2.34 (m, 4H), 2.89 (bs, 2H), 3.50 (bs, 2H), 4.16 (t, 2H), 6.78 (dd, J=8.4 Hz, 2.26 Hz, 1H), 6.97 (d, J=2.36 Hz, 1H), 7.29 (m, 2H), 7.39 (m, 2H), 7.77 (d, J=7.6 Hz, 1H). FABMS: m/z 407 (M+1).

### EXAMPLE NO. 30

### 9-Hydroxy-12-[5-(pyrrolidine-1-yl) pentyl]-6,7-dihydro-12H-benzothiepino [5,4-b]indole

To a solution of 9-methoxy-12-[5-(pyrrolidine-1-yl) pentyl]-6,7-dihydro-12H-benzothiepino [5,4-b]indole (0.5 g, 1.12 mM) in dry DCM (20 ml), BBr₃ (1M soln in DCM, 5.76 ml, 5.56 mM) was added dropwise under nitrogen atmosphere at −10° C. The reaction mixture was stirred for 3 hr. then allowed to attain room temp. The reaction mixture was quenched with methanol and saturated with aq. NaHCO₃ soln. It was extracted with DCM, dried over sodium sulphate and concentrated. The concentrate was chromatographed over silica gel in methanol chloroform (1:20) to afford a white solid, 0.24 g (50%), mp 217-219° C. ¹H NMR (DMSO-D₆) δ: 0.77 (m, 2H), 1.18 (m, 4H), 1.62 (m, 4H), 2.28 (t, 4H), 2.56 (t, 2H), 2.81 (bs, 2H), 3.51 (bs, 2H), 4.10 (t, 2H), 6.50 (dd, J=8.4 Hz, 2.26 Hz, 1H), 6.67 (d, J=2.36 Hz, 1H), 7.11 (m, 2H), 7.36 (m, 2H), 7.56 (d, J=7.6 Hz, 1H), 8.66 (s, 1H, OH). FABMS: m/z 407 (M+1).

### EXAMPLE NO. 31

### 3,9-Dimethoxy-12-[5-chloro pentyl]-6,7-dihydro-12H-benzothiepino [5,4-b]indole

To a suspension of NaH (60% suspension in oil)(0.48 g, 20 mM) in 10 ml dry DMF was added 3,9-dimethoxy-6,7-dihydro-12H-benzothiepino [5,4-b]indole (3.11 g, 10 mM, dissolved in 20 ml dry DMF) at 0° C. under nitrogen atmosphere with stirring. After 15 min, 1-bromo-5-chloro pentane (2.77 g, 15 mM, dissolved in 20 ml DMF) was added dropwise and continued stirring at room temperature for 1.5 hr. The reaction mixture was poured into water, extracted with ethyl acetate and dried over sodium sulphate. The concentrate was chromatographed on silica gel using ethyl acetate/hexane (1:20) to yield an oil, 3.74 g (90%). ¹H NMR (CDCl₃) δ: 1.16 (m, 2H), 1.63 (m, 4H), 2.99 (bs, 2H), 3.35 (t, 2H), 3.54 (bs, 2H), 3.87 (s, 3H, OCH₃), 3.88 (s, 3H, OCH₃), 4.18 (t, 2H), 6.87 (dd, J=2.36, 8.8 Hz, 1H), 6.97 (d, J=2.6 Hz, 1H), 7.01 (m, 2H), 7.24 (s, 1H), 7.33 (dd, J=2.4, 8.8 Hz 1H). FABMS: m/z 416 (M+1).

### EXAMPLE NO. 32

### 3,9-Dimethoxy-12-[5-(piperidine-1-yl) pentyl]-6,7-dihydro-12H-benzothiepino [5,4-b]indole

A solution of 3,9-dimethoxy-12-(5-chloropentyl)-6,7-dihydro-12H-benzothiepino [5,4-b]indole (1.0 g, 2.4 mM), piperidine (1.0 ml) and tetra butyl ammonium iodide (20 mg) in dry DMF (20 ml) was heated at 70-75° C. with stirring for 7 hr. Reaction mixture was diluted with ethyl acetate, washed with water, dried over sodium sulphate and concentrated. The concentrate was chromatographed on basic alumina using ethyl acetate/hexane (1:50) to yield an oil, 0.94 g (85%). ¹H NMR (CDCl₃) 6:1.07 (m, 2H), 1.29 (m, 2H), 1.38 (m, 2H), 1.56 (m, 6H), 2.07 (t, 2H), 2.26 (m, 4H), 2.99 (bs, 2H), 3.53 (bs, 2H), 3.87 (s, 3H, OCH₃), 3.88 (s, 3H, OCH₃), 4.19 (t, 2H), 6.87 (d, J=2.6 Hz, 1H), 7.01 (m, 2H), 7.24 (s, 1H), 7.33 (dd, J=2.4, 8.8 Hz, 1H). EIMS: m/z 464 (M+).

### EXAMPLE NO. 33

### 3,9-Dimethoxy-12-[5-(pyrrolidine-1-yl) pentyl]-6,7-dihydro-12H-benzothiepino [5,4-b]indole

A solution of 3,9-dimethoxy-12-(5-chloropentyl)-6,7-dihydro-12H-benzothiepino [5,4-b]indole (1.0 g, 2.4 mM), pyrrolidine (1.0 ml) and tetra butyl ammonium iodide (20 mg) in dry DMF (20 ml) was heated at 70-75° C. with stirring for 7 hr. Reaction mixture was diluted with ethyl acetate, washed with water, dried over sodium sulphate and concentrated. The concentrate was chromatographed on basic alumina using ethyl acetate/hexane (1:50) to yield an oil, 0.89 g (82%). ¹H NMR (CDCl₃) 6:1.14 (m, 2H), 1.33 (m, 2H), 1.69 (m, 2H), 1.75 (m, 4H), 2.21 (t, 2H), 2.36 (m, 4H), 2.99 (bs, 2H), 3.54 (bs, 2H), 3.87 (s, 3H, OCH₃), 3.88 (s, 3H, OCH₃), 4.19 (t, 2H), 6.87 (d, J=2.6 Hz, 1H), 7.01 (m, 2H), 7.24 (s, 1H), 7.33 (dd, J=2.4, 8.8 Hz, 1H). EIMS: m/z 450 (M+).

### EXAMPLE NO. 34

### 3,9-Dimethoxy-12-[5-(N-butyl methyl amino) pentyl]-6,7-dihydro-12H-benzothiepino [5,4-b]indole

A solution of 3,9-dimethoxy-12-(5-chloropentyl)-6,7-dihydro-12H-benzothiepino [5,4-b]indole (1.0 g, 2.4 mM), N-methyl butyl amine (1.0 ml) and tetra butyl ammonium iodide (20 mg) in dry DMF (20 ml) was heated at 70-75° C. with stirring for 7 hr. Reaction mixture was diluted with ethyl acetate, washed with water, dried over sodium sulphate and concentrated. The concentrate was chromatographed on basic alumina using ethyl acetate/hexane (1:50) to yield an oil, 0.95 g (85%). ¹H NMR (CDCl₃) δ: 0.92 (m, 5H), 1.09 (m, 2H), 1.36 (m, 6H), 1.61 (m, 2H), 2.11 (s, 3H), 2.25 (m, 4H), 2.99 (bs, 2H), 3.52 (bs, 2H), 3.87 (s, 3H, OCH₃), 3.88 (s, 3H, OCH₃), 4.19 (t, 2H), 6.87 (d, J=2.6 Hz, 1H), 7.01 (m, 2H), 7.24 (s, 1H), 7.33 (dd, J=2.4, 8.8 Hz, 1H). FABMS: m/z 467 (M+1).

### EXAMPLE NO. 35

### 3,9-Dihydroxy-12-[5-(piperidine-1-yl)pentyl]-6,7-dihydro-12H-benzothiepino [5,4-b]indole

To a solution of 3,9-dimethoxy-12-[5-(piperidine-1-yl) pentyl]-6,7-dihydro-12H-benzothiepino [5,4-b]indole (0.6 g, 1.29 mM) in dry DCM (20 ml), BBr₃ (1M soln in DCM, 6.46 ml, 6.4 mM) was added dropwise under nitrogen atmosphere at −10° C. The reaction mixture was stirred for 3 hr. then allowed to attain room temp. It was quenched with methanol and saturated with aq. NaHCO₃ solution It was extracted with DCM, dried over sodium sulphate and concentrated. The concentrate was chromatographed over silica gel in methanol chloroform (1:20) to afford a white solid. 0.30 g (53%), mp 195-196° C. ¹H NMR (DMSO-D₆) δ: 0.95 (m, 2H), 1.39 (m, 4H), 1.63 (m, 6H), 2.74 (m, 6H), 3.23 (bs, 2H), 3.42 (bs, 2H), 4.16 (t, 2H), 6.76 (dd, J=2.1, 8.4 Hz, 1H), 6.85 (d, J=1.5 Hz, 1H), 6.91 (dd, J=2.1, 8.4 Hz, 1H), 7.15 (d, J=2.1 Hz, 1H), 7.34 (m, 2H), 8.74 (s, 1H, OH), 9.85 (s, 1H, OH). FABMS m/z: 437 (M+1).

### EXAMPLE NO. 36

### 3,9-Dihydroxy-12-[5-(pyrrolidine-1-yl)pentyl]-6,7-dihydro-12H-benzothiepino [5,4-b]indole

A solution of 3,9-dimethoxy-12-[5-(piperidine-1-yl) pentyl]-6,7-dihydro-12H-benzothiepino [5,4-b]indole (0.6 g, 1.33 mM) in dry DCM (20 ml), BBr₃ (1M soln in DCM, 6.46 ml, 6.4 mM) was added dropwise under nitrogen atmosphere at −10° C. The reaction mixture was stirred for 3 hr. then allowed to attain room temp. It was quenched with methanol and saturated with aq. NaHCO₃ solution It was extracted with DCM, dried over sodium sulphate and concentrated. The concentrate was chromatographed over silica gel in methanol chloroform (1:20) to afford a white solid, 0.24 g (42%), mp 202-204° C. ¹H NMR (DMSO-D₆) δ: 1.06 (m, 2H), 1.46 (m, 4H), 1.86 (m, 4H),) 2.85 (m, 6H) 2.05 (bs, 2H), 3.54 (bs, 2H), 4.16 (t, 2H), 6.71 (dd, J=2.1, 8.4 Hz, 1H), 6.87 (d, J=1.5 Hz, 1H), 6.95 (dd, J=2.1, 8.4 Hz, 1H), 7.17 (d, J=2.1 Hz, 1H), 7.34 (t, 2H), 8.74 (s, 1H, OH), 9.86 (s, 1H, OH). FABMS: m/z 423 (M+1).

### EXAMPLE NO. 37

### 3,9-Dihydroxy-12-[5-(pentylamin-3-yl)pentyl]-6,7-dihydro-12H-benzothiepino [5,4-b]indole

To a solution of 3,9-dimethoxy-12-[5-(pentylamin-3-yl) pentyl]-6,7-dihydro-12H-benzothiepino [5,4-b]indole (0.8 g, 1.71 mM) in dry DCM (20 ml), BBr₃ (1M soln in DCM, 8.55 ml, 8.55 mM) was added dropwise under nitrogen atmosphere at −10° C. The reaction mixture was stirred for 3 hr. then allowed to attain room temp. It was quenched with methanol and saturated with aq. NaHCO₃ solution It was extracted with DCM, dried over sodium sulphate and concentrated. The concentrate was chromatographed over silica gel in methanol chloroform (1:20) to afford a white solid. 0.29 g (38%), mp 134-136° C. ¹H NMR (DMSO-D₆) δ: 0.92 (m, 3H), 1.09 (m, 2H), 1.18-1.56 (m, 8H), 2.60 (s, 3H), 2.80 (m, 4H), 3.02 (bs, 4H), 4.16 (t, 2H), 6.71 (dd, J=2.1, 8.4 Hz, 1H), 6.87 (d, J=1.5 Hz, 1H), 6.95 (dd, J=2.1, 8.4 Hz, 1H), 7.15 (d, J=2.1 Hz, 1H), 7.42 (m, 2H), 8.74 (s, 1H, OH), 9.86 (s, 1H, OH). FABMS: m/z 439 (M+1).

### EXAMPLE NO. 38

### 12-[4-(2-Chloro-ethoxy) benzyl]-6,7-dihydro-12H-benzothiepino [5,4-b]indole

To a suspension of NaH (60% suspension in oil, 0.072 g, 3 mM) in 10 ml dry DMF was added 6,7-dihydro-12H-benzothiepino [5,4-b]indole (0.5 g, 2 mM, dissolved in 30 ml dry DMF) at 0° C. under nitrogen atmosphere with stirring. After 15 min, 4-(2-chloroethoxy) benzyl bromide (0.744 g, 3 mM, dissolved in 20 ml DMF) was added dropwise and stirring was continued at room temperature for 1.5 hr. The reaction mixture was poured into water, extracted with ethyl acetate and dried over sodium sulphate. The concentrate was chromatographed on silica gel using ethyl acetate/hexane (1:20) to yield an oil, 0.62 g (74.6%). ¹H NMR (CDCl₃) δ: 2.99 (bs, 2H), 3.57 (bs, 2H), 3.78 (t, 2H), 4.18 (t, 2H), 5.32 (s, 2H), 6.81 (d, 2H, J=8.64 Hz), 6.99 (d, 2H, J=8.64 Hz), 7.20 (m, 2H), 7.35 (m, 4H), 7.62 (m, 1H), 7.77 (d, 1H, J=6.8 Hz). EIMS m/z: 419 (M+)

### EXAMPLE NO. 39

### 12-[4-(2-Piperidin-1-yl-ethoxy) benzyl]-6,7-dihydro-12H-benzothiepino [5,4-b]indole

A solution of 12-[4-(2-chloro-ethoxy) benzyl]-6,7-dihydro-12H-benzothiepino [5,4-b]indole (0.5 g, 1.12 mM), piperidine (0.5 ml) and tetra butyl ammonium iodide (10 mg) in dry DMF (15 ml) was heated at 70-75° C. with stirring for 7 hr. After cooling the reaction mixture was diluted with ethyl acetate, washed with water, dried over sodium sulphate and concentrated. The concentrate was chromatographed on basic alumina using ethyl acetate/hexane (1:50) to yield an oil, 0.44 g (85%). ¹H NMR (CDCl₃) δ: 1.44 (m, 2H), 1.57 (m, 4H), 2.47 (m, 6H), 3.05 (bs, 2H), 3.57 (bs, 2H), 4.18 (t, 2H), 5.32 (s, 2H), 6.81 (d, 2H, J=8.64 Hz), 6.99 (d, 2H, J=8.64 Hz), 7.20 (m, 2H), 7.35 (m, 4H), 7.62 (m, 1H), 7.77 (d, 1H, J=6.8 Hz). EIMS: m/z 468 (M+).

### EXAMPLE NO. 40

### 12-[4-(3-Chloro propyloxy) benzyl]-6,7-dihydro-12H-benzothiepino [5,4-b]indole

To a suspension of NaH (60% suspension in oil, 0.072 g, 3 mM) in 5 ml dry DMF was added 6,7-dihydro-12H-benzothiepino [5,4-b]indole (0.5 g, 20 mM, dissolved in 5 ml dry DMF) at 0° C. with stirring under nitrogen atmosphere. After 15 min, 4-(2-chloroethoxy) benzyl bromide (0.774 g, 30 mM, dissolved in 10 ml DMF) was added dropwise and stirring was continued at room temperature for 1.5 hr. The reaction mixture was poured into water, extracted with ethyl acetate, dried over sodium sulphate and concentrated. The concentrate was chromatographed on silica gel using ethyl acetate/hexane (1:20) to yield an oil, 0.64 g (75%). ¹H NMR (CDCl₃) δ: 2.22 (m, 2H), 3.02 (bs, 2H), 3.57 (bs, 2H) 3.76 (t, 2H), 4.07 (t, 2H), 5.32 (s, 2H), 6.81 (d, J=8.64 Hz, 2H,), 6.99 (d, J=8.64 Hz, 2H), 7.19 (m, 2H), 7.27 (m, 4H), 7.62 (m, 1H), 7.77 (d, J=6.8 Hz, 1H). EIMS: m/z 433 (M+).

### EXAMPLE NO. 41

### 12-[4-(3-Piperidin-1-yl-propyloxy)benzyl]-6,7-dihydro-12H-benzothiepino [5,4-b]indole

A solution of 12-[4-(3-chloro-propyloxy)benzyl]-6,7-dihydro-12H-benzothiepino [5,4-b]indole (0.5 g, 1.15 mM), piperidine (0.5 ml), tetrabutyl ammonium iodide (10 mg) in dry DMF (15 ml) was heated at 70-75° C. with stirring for 7 hr. After cooling the reaction mixture was diluted with ethyl acetate, washed with water, dried over sodium sulphate and concentrated. The concentrate was chromatographed on basic alumina using ethyl acetate/hexane (1:50) to yield a white solid, 0.47 g (85%), mp 70-71° C. ¹H NMR (CDCl₃): 1.44 (m, 2H), 1.57 (m, 4H), 1.96 (m, 2H), 2.47 (m, 6H), 3.05 (bs, 2H), 3.57 (bs, 2H), 3.97 (t, 2H), 5.32 (s, 2H), 6.80 (d, J=8.64 Hz, 2H), 6.98 (d, J=8.64 Hz, 2H), 7.18 (m, 2H), 7.29 (m, 4H), 7.62 (m, 1H), 7.77 (d, 1H, J=6.8 Hz). EIMS m/z: 482 (M+).

### EXAMPLE NO. 42

### 12-[4-(5-Chloro pentyloxy) benzyl]-6,7-dihydro-12H-benzothiepino [5,4-b]indole

To a suspension of NaH (60% suspension in oil, 0.072 g, 3 mM) in 5 ml dry DMF was added 6,7-dihydro-12H-benzothiepino [5,4-b]indole (0.5 g, 2 mM, dissolved in 5 ml dry DMF) at 0° C. under nitrogen atmosphere with stirring. After 15 min, 4-(5-chloropentyloxy) benzyl bromide (0.87 g, 3 mM, dissolved in 5 ml DMF) was added dropwise and stirring was continued at room temperature for 1.5 hr. The reaction mixture was poured into water, extracted with ethyl acetate, dried over sodium sulphate and concentrated. The concentrate was chromatographed on silica gel using ethyl acetate/hexane (1:20) to yield an oil, 0.78 g (85%). ¹H NMR (CDCl₃) δ: 1.67 (m, 2H), 1.91 (m, 4H), 3.06 (bs, 2H), 3.51 (bs, 2H) 3.57 (t, 2H), 3.93 (t, 2H), 5.32 (s, 2H), 6.79 (d, J=8.64 Hz, 2H), 6.98 (d, J=8.64 Hz, 2H), 7.18 (m, 2H), 7.35 (m, 4H), 7.65 (m, 1H), 7.77 (d, J=6.8 Hz, 1H). EIMS: m/z 461 (M+).

### EXAMPLE NO. 43

### 12-[4-(5-Piperidin-1-yl-pentyloxy) benzyl]-6,7-dihydro-12H-benzothiepino [5,4-b]indole

A solution of 12-[4-(5-chloro-propyloxy) benzyl]-6,7-dihydro-12H-benzothiepino [5,4-b]indole (0.5 g, 1.08 mM), piperidine (0.5 ml) and tetra butyl ammonium iodide (10 mg) in dry DMF (15 ml) was heated at 70-75° C. with stirring for 7 hr. After cooling the reaction mixture was diluted with ethyl acetate, washed with water, dried over sodium sulphate and concentrated. The concentrate was chromatographed on basic alumina using ethyl acetate/hexane (1:50) to yield an oil, 0.46 g (83%). ¹H NMR (CDCl₃): 1.44 (m, 4H), 1.57 (m, 6H), 1.79 (m, 2H), 2.36 (m, 6H), 3.05 (bs, 2H), 3.60 (bs, 2H), 3.91 (t, 2H), 5.31 (s, 2H), 6.79 (d, J=8.64 Hz, 2H), 6.98 (d, J=8.64 Hz, 2H), 7.19 (m, 2H), 7.35 (m, 4H), 7.62 (m, 1H), 7.77 (d, J=6.8 Hz, 1H). FABMS: m/z 511 (M+1).

### EXAMPLE NO. 44

### 6,7-Dihydro-12H-benzoxepino[5,4-b]indole

A solution of benzoxepine (0.162 g, 1 mM), phenyl hydrazine (0.108 g, 1 mM), glacial acetic acid (few drops) in ethanol (5 ml) was refluxed on water bath for 5-6 hr. It was cooled, filtered the solid. The crude hydrazone was used as such without further purification as it turned coloured on standing at room temp.

A solution of benzoxepine phenyl hydrazone in ethanol (4 ml) and 20% aq. HCl (3 ml) and the reaction mixture was refluxed for 12 hr. On completion, the reaction mixture was poured on ice, neutralized with 5% aq. NaOH. The precipitated solid was filtered, dried and crystallized in methanol to afford an off white solid, 0.2 g (85%), mp 161° C. ¹H NMR (CDCl₃) δ: 3.36 (t, 2H), 4.47 (t, 2H), 7.09-7.29 (m, 5H), 7.39 (d, J=7.6 Hz, 1H), 7.58 (m, 2H), 8.18 (s, 1H). EIMS: m/z 235 (M+).

### EXAMPLE NO. 45

### 12-[2-(Piperidine-1-yl) ethyl]-6,7-dihydro-12H-benzoxepino[5,4-b]indole

To a suspension of NaH (60% suspension in oil, 0.100 g, 4.2 mM) in 5 ml dry DMF was added 6,7-dihydro-12H-benzoxepino[5,4-b]indole (0.5 g, 2.1 mM, dissolved in 5 ml dry DMF) at 0° C. under nitrogen atmosphere with stirring. After 15 min, 1-(2-chloroethyl) piperidine hydrochloride (0.584 mg, 3.17 mM, dissolved in 15 ml DMF) was added dropwise and stirring was continued at room temperature for 3 hr. The reaction mixture was poured into water, extracted with ethyl acetate, dried over sodium sulphate and concentrated. The concentrate was chromatographed on basic alumina using ethyl acetate/hexane (1:50) to yield a white solid, 0.55 g (76%), mp 103° C. ¹H NMR (CDCl₃) δ: 1.43 (m, 2H), 1.62 (m, 4H), 2.41 (m, 4H), 2.73 (t, 2H), 3.08 (t, 2H), 4.43 (t, 2H), 4.62 (t, 2H), 7.15-7.22 (m, 5H), 7.44 (d, J=7.6 Hz, 1H), 7.62 (m, 2H). EIMS: m/z 346 (M+).

### EXAMPLE NO. 46

### 12-[3-Chloro propyl]-6,7-dihydro-12H-benzoxepino[5,4-b]indole

To a suspension of NaH (60% suspension in oil, 0.072 g, 3 mM) in 5 ml dry DMF was added 6,7-dihydro-12H-benzoxepino[5,4-b]indole (0.47 g, 2 mM, dissolved in 5 ml dry DMF) at 0° C. under nitrogen atmosphere with stirring. After 15 min, 1-bromo-3-chloro propane (0.47 g, 3 mM, dissolved in 15 ml DMF) was added dropwise and continued stirring at room temperature for 3 hr. The reaction mixture was poured into water, extracted with ethyl acetate, dried over sodium sulphate and concentrated. The concentrate was chromatographed on silica gel using ethyl acetate/hexane (1:20) to yield an oil, 0.54 g (88%). ¹H NMR (CDCl₃) δ: 2.02 (m, 2H), 3.07 (t, 2H), 3.37 (t, 2H), 4.53 (t, 2H), 4.61 (t, 2H), 7.15-7.22 (m, 5H), 7.44 (d, J=7.6 Hz, 1H), 7.62 (m, 2H). EIMS: m/z 311 (M+).

### EXAMPLE NO. 47

### 12-[3-(Piperidine-1-yl) propyl]-6,7-dihydro-12H-benzoxepino[5,4-b]indole

A solution of 12-(3-chloropropyl)-6,7-dihydro-12H-benzoxepino[5,4-b]indole (0.5 g, 1.6 mM), piperidine (0.5 ml) and tetra butyl ammonium iodide (10 mg) in dry DMF (15 ml) was heated at 70-75° C. with stirring for 7 hr. Reaction mixture was diluted with ethyl acetate, washed with water, dried over sodium sulphate and concentrated. The concentrate was chromatographed on basic alumina using ethyl acetate/hexane (1:50) to yield a white solid, 540 mg (95%), mp 65-66° C. ¹H NMR (CDCl₃) δ: 1.40 (m, 2H), 1.51 (m, 4H), 1.98 (m, 2H), 2.24 (m, 6H), 3.07 (t, 2H), 4.36 (t, 2H), 4.61 (t, 2H), 7.15-7.22 (m, 5H), 7.44 (d, J=7.6 Hz, 1H), 7.62 (m, 2H). FABMS: m/z 361 (M+1).

### EXAMPLE NO. 48

### 12-(5-Chloropentyl-6,7-dihydro-12H-benzoxepino[5,4-b]indole

To a suspension of NaH (60% suspension in oil, 0.48 g, 20 mM) in 20 ml dry DMF was added 6,7-dihydro-12H-benzoxepino[5,4-b]indole (2.35 g, 10 mM, dissolved in 20 ml dry DMF) at 0° C. under nitrogen atmosphere with stirring. After 15 min, 1-bromo-5-chloro pentane (3.7 g, 20 mM, dissolved in 20 ml DMF) was added dropwise and continued stirring at room temperature for 1.5 hr. The reaction mixture was poured into water and extracted with ethyl acetate, dried over sodium sulphate. The concentrate was chromatographed on silica gel using ethyl acetate/hexane (1:20) to yield an oil, 3.50 g (90%). ¹H NMR (CDCl₃) δ: 1.29 (m, 2H), 1.63 (m, 4H), 3.07 (t, 2H), 3.37 (t, 2H), 4.53 (t, 2H), 4.61 (t, 2H), 7.15-7.22 (m, 5H), 7.44 (d, J=7.6 Hz, 1H), 7.62 (m, 2H). EIMS: m/z 390 (M+).

### EXAMPLE NO. 49

### 12-[5-(Piperidine-1-yl) pentyl]-6,7-dihydro-12H-benzoxepino[5,4-b]indole

A solution of 12-(5-chloropentyl)-6,7-dihydro-12H-benzoxepino[5,4-b]indole (0.68 g, 2 mM), piperidine (1 ml) and tetra butyl ammonium iodide (10 mg) in dry DMF (20 ml) was heated with stirring at 70-75° C. for 7 hr. Reaction mixture was diluted with ethyl acetate, washed with water, dried over sodium sulphate and concentrated. The concentrate was chromatographed on basic alumina using ethyl acetate/hexane (1:50) to yield an oil, 0.74 g (95%). ¹H NMR (CDCl₃) δ: 1.26 (m, 2H), 1.43 (m, 4H), 1.57 (m, 4H), 2.18 (t, 2H), 2.18 (t, 2H), 2.31 (m, 4H), 3.07 (t, 2H), 4.27 (t, 2H), 4.61 (t, 2H), 7.15-7.22 (m, 5H), 7.44 m, 2H), 7.62 (d, J=7.6 Hz, 1H). FABMS: m/z 389 (M+1).

### EXAMPLE NO. 50

### 12-(6-Chloro hexyl)-6,7-dihydro-12H-benzoxepino[5,4-b]indole

To a suspension of NaH (60% suspension in oil, 0.048 g, 2 mM) in 5 ml dry DMF was added 6,7-dihydro-12H-benzoxepino[5,4-b]indole (0.235 g, 1 mM, dissolved in 5 ml dry DMF) at 0° C. under nitrogen atmosphere with stirring. After 15 min, 1-bromo-6-chloro hexane (0.4 g, 2 mM, dissolved in 5 ml DMF) was added dropwise and continued stirring at room temperature for 1.5 hr. The reaction mixture was poured into water and extracted with ethyl acetate, dried over sodium sulphate and concentrated. The concentrate was chromatographed on silica gel using ethyl acetate/hexane (1:20) to yield an oil, 0.34 g (95%). ¹H NMR (CDCl₃) δ: 1.33 (m, 4H), 1.64 (m, 2H), 1.76 (m, 2H), 3.07 (t, 2H), 3.42 (t, 2H), 4.31 (t, 2H), 4.61 (t, 2H), 7.15-7.22 (m, 5H), 7.44 (d, J=7.6 Hz, 1H), 7.62 (m, 2H). FABMS: m/z 354 (M+1).

### EXAMPLE NO. 51

### 12-[6-(Piperidine-1-yl)hexyl]-6,7-dihydro-12H-benzoxepino[5,4-b]indole

A solution of 12-(5-chloropentyl)-6,7-dihydro-12H-benzoxepino[5,4-b]indole (0.33 g, 0.93 mM), piperidine (0.5 ml) and tetra butyl ammonium iodide (10 mg) in dry DMF (10 ml) was heated at 70-75° C. with stirring for 7 hr. Reaction mixture was diluted with ethyl acetate, washed with water, dried over sodium sulphate and concentrated. The concentrate was chromatographed on basic alumina using ethyl acetate/hexane (1:50) to yield an off white solid, 0.35 g (95%), mp 110° C. ¹H NMR (CDCl₃) δ: 1.24 (m, 4H), 1.43 (m, 2H), 1.59 (m, 4H), 1.75 (m, 4H) 2.23 (t, 2H), 2.32 (m, 4H), 3.07 (t, 2H), 4.27 (t, 2H), 4.61 (t, 2H), 7.15-7.22 (m, 5H), 7.44 (m, 2H), 7.62 (d, J=7.6 Hz, 1H). FABMS: m/z 403 (M+1).

### EXAMPLE NO. 52

### 3-Methoxy-6,7-dihydro-12H-benzoxepino[5,4-b]indole

A solution of 8-methoxy benzoxepine (0.192 g, 1 mM), phenyl hydrazine (0.108 g, 1 mM), glacial acetic acid (few drops) in ethanol (5 ml) was refluxed on water bath for 5-6 hr. It was cooled, filter the solid. The crude hydrazone was used as such without further purification as it turned coloured on standing at room temp.

A solution of 8-methoxy benzoxepine phenyl hydrazone in ethanol (4 ml) and 20% aq. HCl (3 ml) and the reaction mixture was refluxed for 12 hr. On completion reaction mixture was poured on ice, neutralized with S % aq. NaOH. and the precipitated solid was filtered. It was crystallized in methanol to afford an off white solid, 0.22 g (82%), mp 167° C. ¹H NMR (CDCl₃) δ: 3.18 (t, 2H), 3.81 (s, 3H, OCH₃), 4.46 (t, 2H), 6.72 (m, 2H), 7.19 (m, 2H), 7.32 (d, J=7.6 Hz, 1H), 7.48 (m, 2H), 8.07 (s, 1H). FABMS: m/z 266 (M+1).

### EXAMPLE NO. 53

### 3-Methoxy-12-(5-bromo pentyl)-6,7-dihydro-12H-benzoxepino[5,4-b]indole

To a suspension of NaH (60% suspension in oil, 0.36 g, 15 mM) in 10 ml dry DMF was added 3-methoxy-6,7-dihydro-12H-benzothiepino[5,4-b]indole (2.65 g, 10 mM, dissolved in 20 ml dry DMF) at 0° C. under nitrogen atmosphere with stirring. After 15 min, 1,5-dibromo pentane (4.6 g, 20 mM, dissolved in 20 ml DMF) was added dropwise, and continued stirring at room temperature for 1.5 hr. The reaction mixture was poured into water extracted with ethyl acetate, dried over sodium sulphate and concentrated. The concentrate was chromatographed on silica gel using ethyl acetate/hexane (1:20) to yield an oil, 2.68 g (65%). ¹H NMR (CDCl₃) δ: 1.35 (m, 2H), 1.77 (m, 4H), 3.06 (t, 2H), 3.30 (t, 2H), 3.85 (s, 3H), 4.27 (t, 2H), 4.60 (t, 2H), 6.82 (m, 2H), 7.22 (m, 2H), 7.38 (m, 2H), 7.56 (d, J=7.8 Hz, 1H), FABMS: m/z 415 (M+1).

### EXAMPLE NO. 54

### 3-Methoxy-12-[5-(piperidine-1-yl) pentyl]-6,7-dihydro-12H-benzothiepino [5,4-b]indole

A solution of 3-methoxy-12-(5-bromopentyl)-6,7-dihydro-12H-benzoxepino[5,4-b]indole (1.05 g, 2.6 mM), piperidine (1.0 ml) and tetra butyl ammonium iodide (20 mg) in dry DMF (20 ml) was heated at 70-75° C. with stirring for 4 hr. Reaction mixture was diluted with ethyl acetate, washed with water, dried over sodium sulphate and concentrated. The concentrate was chromatographed on basic alumina using ethyl acetate/hexane (1:50) to yield an oil, 0.87 g (85%). ¹H NMR (CDCl₃) δ: 1.27 (m, 2H), 1.45 (m, 2H), 1.56 (m, 4H), 1.83 (m, 4H), 2.20 (t, 2H), 2.31 (m, 4H), 3.07 (t, 2H), 3.85 (s, 3H, OCH₃), 4.25 (t, 2H), 4.61 (t, 2H), 6.81 (m, 2H), 7.22 (m, 2H), 7.41 (m, 2H), 7.56 (d, J=7.8 Hz, 1H). FABMS: m/z 419 (M+1).

### EXAMPLE NO. 55

### 12-[4-(2-Chloroethoxy) benzyl]-6,7-dihydro-12H-benzoxepino[5,4-b]indole

To a suspension of NaH (60% suspension in oil, 0.072 g, 3 mM) in 10 ml dry DMF was added 6,7-dihydro-12H-benzoxepino[5,4-b]indole (0.47 g, 2 mM, dissolved in 5 ml dry DMF) at 0° C. under nitrogen atmosphere with stirring. After 15 min, 4-(2-chloroethoxy) benzyl bromide (0.744 g, 3 mM, dissolved in 10 ml DMF) was added dropwise and continued stirring at room temperature for 1.5 hr. The reaction mixture was poured into water, extracted with ethyl acetate, dried over sodium sulphate and concentrated. The concentrate was chromatographed on silica gel using ethyl acetate/hexane (1:20) to yield an off white solid, 0.64 g (80%). ¹H NMR (CDCl₃) δ: 3.21 (t, 2H), 3.82 (t, 2H), 4.23 (t, 2H), 4.63 (t, 2H), 5.32 (s, 2H), 6.81 (d, J=8.64 Hz, 2H), 7.05 (m, 2H), 7.15-7.35 (m, 5H), 7.63 (dd, J=1.8 Hz, 7.6 Hz, 1H). EIMS: m/z 403 (M+1).

### EXAMPLE NO. 56

### 12-[4-(2-Piperidin-1-yl-ethoxy) benzyl]-6,7-dihydro-12H-benzoxepino[5,4-b]indole

A solution of 12-[4-(2-chloroethoxy) benzyl]-6,7-dihydro-12H-benzoxepino[5,4-b]indole (0.6 g, 1.4 mM), piperidine (0.5 ml) and tetra butyl ammonium iodide (10 mg) in dry DMF (15 ml) was heated at 70-75° C. with stirring for 7 hr. After cooling the reaction mixture was diluted with ethyl acetate, washed with water, dried over sodium sulphate and concentrated. The concentrate was chromatographed on basic alumina using ethyl acetate/hexane (1:50) to yield an off white solid, 0.57 g (85%). ¹H NMR (CDCl₃) δ: 1.45 (m, 2H), 1.65 (m, 4H), 2.52 (m, 4H), 2.78 (t, 2H), 3.21 (t, 2H), 4.10 (t, 2H), 4.62 (t, 2H), 5.34 (s, 2H), 6.88 (d, 2H, J=8.8 Hz), 7.05 (m, 2H), 7.15-7.35 (m, 5H), 7.63 (dd, J=1.8 Hz, 7.6 Hz, 1H). FABMS: m/z 453 (M+1).

### EXAMPLE NO. 57

### 12-[4-(3-Chloro propyloxy) benzyl]-6,7-dihydro-12H-benzoxepino[5,4-b]indole

To a suspension of NaH (60% suspension in oil, 0.036 g, 1.5 mM) in 5 ml dry DMF was added 6,7-dihydro-12H-benzoxepino[5,4-b]indole (0.235 g, 1 mM, dissolved in 5 ml dry DMF) at 0° C. under nitrogen atmosphere with stirring. After 15 min, 4-(2-chloro propyloxy) benzyl bromide (0.526 g, 2 mM, dissolved in 10 ml DMF) was added dropwise and continued stirring at room temperature for 1.5 hr. The reaction mixture was poured into water, extracted with ethyl acetate, dried over sodium sulphate and concentrated. The concentrate was chromatographed on silica gel using ethyl acetate/hexane (1:20) to yield an off white solid, 0.29 g (70%). ¹H NMR (CDCl₃) δ: 2.28 (m, 2H), 3.21 (t, 2H), 3.77 (t, 2H), 4.12 (t, 2H), 4.63 (t, 2H), 5.35 (s, 2H), 6.88 (d, J=8.8 Hz, 2H), 7.05 (m, 2H), 7.15-7.35 (m, 5H), 7.63 (dd, J=1.8 Hz, 7.6 Hz, 1H), EIMS: m/z 417 (M+).

### EXAMPLE NO. 58

### 12-[4-(3-Piperidin-1-yl-propyloxy) benzyl]-6,7-dihydro-12H-benzoxepino[5,4-b]indole

A solution of 12-[4-(3-chloro-propyloxy) benzyl]-6,7-dihydro-12H-benzoxepino[5,4-b]indole (0.29 g, 0.7 mM), piperidine (0.5 ml) and tetra butyl ammonium iodide (10 mg) in dry DMF (15 ml) was heated at 70-75° C. with stirring for 7 hr. After cooling the reaction mixture was diluted with ethyl acetate, washed with water, dried over sodium sulphate and concentrated. The concentrate was chromatographed on basic alumina using ethyl acetate/hexane (1:50) to yield a white solid, 0.28 g (87.5%), mp 129-130° C. ¹H NMR (CDCl₃): 1.58 (m, 2H), 1.61 (m, 4H), 2.0 (m, 2H), 2.50 (m, 6H), 3.21 (t, 2H), 4.01 (t, 2H), 4.63 (t, 2H), 5.35 (s, 2H), 6.88 (d, J=8.8 Hz, 2H), 7.05 (m, 2H), 7.15-7.35 (m, 5H), 7.63 (dd, J=1.8 Hz, 7.6 Hz, 1H). FABMS: m/z 467 (M+1).

### EXAMPLE NO. 59

### 12-[4-(5-Chloro pentyloxy) benzyl]-6,7-dihydro-12H-benzoxepino[5,4-b]indole

To a suspension of NaH (60% suspension in oil, 0.036 g, 1.5 mM) in 5 ml dry DMF was added 6,7-dihydro-12H-benzoxepino[5,4-b]indole (0.235 g, 1 mM, dissolved in 5 ml dry DMF) at 0° C. under nitrogen atmosphere with stirring. After 15 min, 4-(5-chloro pentyloxy) benzyl bromide (0.43 g, 1.5 mM, dissolved in 5 ml DMF) was added dropwise, and continued stirring at room temperature for 1 hr. The reaction mixture was poured into water and extracted with ethyl acetate, dried over sodium sulphate and concentrated. The concentrate was chromatographed on silica gel using ethyl acetate/hexane (1:20) to yield an off white solid, 0.35 g (80%). ¹H NMR (CDCl₃) δ: 1.65 (m, 2H), 1.88 (m, 4H), 3.21 (t, 2H), 3.59 (t, 2H), 4.01 (t, 2H), 4.63 (t, 2H), 5.35 (s, 2H), 6.88 (d, J=8.8 Hz, 2H), 7.05 (m, 2H), 7.15-7.35 (m, 5H), 7.63 (dd, J=1.8 Hz, 7.6 Hz, 1H). FABMS: m/z 446 (M+1).

### EXAMPLE NO. 60

### 12-[4-(5-Piperidin-1-yl-pentyloxy) benzyl]-6,7-dihydro-12H-benzothiepino [5,4-b]indole

A solution of 12-[4-(5-chloro-pentyloxy) benzyl]-6,7-dihydro-12H-benzoxepino[5,4-b]indole (0.3 g, 0.74 mM), piperidine (0.5 ml) and tetra butyl ammonium iodide (10 mg) in dry DMF (15 ml) was heated at 70-75° C. with stirring for 7 hr. After cooling the reaction mixture was diluted with ethyl acetate, washed with water, dried over sodium sulphate and concentrated. The concentrate was chromatographed on basic alumina using ethyl acetate/hexane (1:50) to yield oil, 0.24 g (68.5%). ¹H NMR (CDCl₃) δ: 1.45 (m, 2H), 1.61 (m, 6H), 1.85 (m, 4H), 2.39 (m, 6H), 3.21 (t, 2H), 3.93 (t, 2H), 4.63 (t, 2H), 5.34 (s, 2H), 6.88 (d, J=8.8 Hz, 2H), 7.05 (m, 2H), 7.15-7.35 (m, 5H), 7.63 (dd, J=1.8 Hz, 7.6 Hz, 1H), FABMS: m/z 523 (M+1).

### EXAMPLE NO. 61

### 5,6,7,12-Tetrahydro-benzo [6,7]cyclohepta[1,2-b]indole

A solution of 6,7,8,9-tetrahydro-benzocyclohepten-5-one (0.16 g, 1 mM), phenyl hydrazine (0.108 g, 1 mM) and glacial acetic acid (few drops) in ethanol (5 ml) was refluxed on water bath for 5-6 hr. It was then cooled and the precipitated solid was filtered. The crude hydrazone was used as such without further purification as it turned colored on standing at room temp.

A solution of 6,7,8,9-tetrahydro-benzocyclohepten-5-one phenyl hydrazone in ethanol (4 ml) and 20% aq. HCl (3 ml) was refluxed for 12 hr. It was poured on ice and neutralized with 5% aq. NaOH. The precipitated solid was filtered and crystallized in methanol to afford an off white solid, 0.20 g (87.23%), mp 96-97° C. ¹H NMR (CDCl₃) δ: 2.17 (t, CH₂), 2.92 (t, CH₂), 3.13 (t, 2H), 7.08-7.53 (m, 6H), 7.57 (d, J=7.2 Hz, 1H), 8.03 (s, 1H). FABMS: m/z 234 (M+1).

### EXAMPLE NO. 62

### 12-(5-Bromopentyl)-5,6,7,12-tetrahydro-benzo [6,7]cyclohepta[1,2-b]indole

To a suspension of NaH (60% suspension in oil, 0.52 g, 12.9 mM) in 20 ml dry DMF was added 5,6,7,12-tetrahydro-benzo[6,7]cyclohepta[1,2-b]indole (2.0 g, 8.6 mM, dissolved in 20 ml dry DMF) at 0° C. under nitrogen atmosphere with stirring. After 15 min, 1,5-dibromo pentane (5.93 g, 26 mM, dissolved in 20 ml DMF) was added dropwise and continued stirring at room temperature for 1.5 hr. The reaction mixture was poured into water, extracted with ethyl acetate, dried over sodium sulphate and concentrated. The concentrate was chromatographed on silica gel using ethyl acetate/hexane (1:20) to yield an oil, 2.54 g (77.4%). ¹H NMR (CDCl₃) δ: 1.31 (m, 2H), 1.72 (m, 4H), 2.31 (m, 2H), 2.66 (m, 4H), 3.25 (t, 2H), 4.33 (t, 2H), 7.08-7.53 (m, 6H), 7.57 (d, J=7.2 Hz, 1H), 8.03 (s, 1H). FABMS: m/z 383 (M+1).

### EXAMPLE NO. 63

### 12-[5-(Piperidine-1-yl)-pentyl)]-5,6,7,12-tetrahydro-benzo [6,7]cyclohepta[1,2-b]indole

A solution of 12-(5-bromopentyl)-5,6,7,12-tetrahydro-benzo[6,7]cyclohepta[1,2-b]indole (0.5 g, 1.3 mM), piperidine (1.0 ml) and tetra butyl ammonium iodide (10 mg) in dry DMF (20 ml) was heated at 70-75° C. with stirring for 7 hr. On completion, the reaction mixture was diluted with ethyl acetate, washed with water, dried over sodium sulphate and concentrated. The concentrate was chromatographed on basic alumina using ethyl acetate/hexane (1:50) to yield an oil, 0.44 g (88%). ¹H NMR (CDCl₃) δ: 1.12 (m, 2H), 1.29 (m, 2H), 1.37 (m, 4H) 1.56 (m, 4H), 1.69 (m, 2H), 2.13 (t, 2H), 2.27 (m, 4H), 2.65 (m, 4H), 4.30 (t, 2H), 7.08-7.53 (m, 6H), 7.57 (d, J=7.2 Hz, 1H). FABMS: m/z 387 (M+1).

### EXAMPLE NO. 64

### 12-[5-(Pyrrolidine-1-yl)-pentyl)]-5,6,7,12-tetrahydro-benzo [6,7]cyclohepta[1,2-b]indole

A solution of 12-(5-bromopentyl)-5,6,7,12-tetrahydro-benzo[6,7]cyclohepta[1,2-b]indole (0.5 g, 1.3 mM), pyrrolidine (1.0 ml) and tetra butyl ammonium iodide (10 mg) in dry DMF (20 ml) was heated at 70-75° C. with stirring for 7 hr. On completion, the reaction mixture was diluted with ethyl acetate, washed with water, dried over sodium sulphate and concentrated. The concentrate was chromatographed on basic alumina using ethyl acetate/hexane (1:50) to yield an oil, 0.36 g (74%). ¹H NMR (CDCl₃) δ: 1.25 (m, 2H), 1.28 (m, 4H), 1.64 (m, 4H), 2.23 (t, 2H), 2.32 (m, 4H), 2.13 (t, 2H), 2.27 (m, 4H), 2.65 (m, 4H), 4.30 (t, 2H), 7.08-7.53 (m, 6H), 7.57 (d, J=7.2 Hz, 11H). FABMS: m/z 373 (M+1)

### EXAMPLE NO. 65

### 12-[5-(N-butyl methyl amino)-pentyl)]-5,6,7,12-tetrahydro-benzo [6,7]cyclohepta[1,2-b]indole

A solution of 12-(5-bromopentyl)-5,6,7,12-tetrahydro-benzo[6,7]cyclohepta[1,2-b]indole (0.5 g, 1.3 mM), N-methyl butyl amine (1.0 ml) and tetra butyl ammonium iodide (10 mg) in dry DMF (20 ml) was heated at 70-75° C. with stirring for 7 hr. On completion, the reaction mixture was diluted with ethyl acetate, washed with water, dried over sodium sulphate and concentrated. The concentrate was chromatographed on basic alumina using ethyl acetate/hexane (1:50) to yield a light yellow powder, 0.41 g (80.8%), mp 169-170° C. ¹H NMR (CDCl₃) δ: 0.92 (t, 3H), 1.09 (m, 2H), 1.41 (m, 2H), 1.68 (m, 6H),) 2.28 (m, 2H), 2.54-2.78 (m, 8H), 4.30 (t, 2H), 7.08-7.53 (m, 6H), 7.57 (d, J=7.2 Hz, 1H). FABMS: m/z 375 (M+1).

Biological Evaluation

The compounds of the present invention were evaluated for use for the prevention or treatment of symptoms of estrogen deficiency or deprivation including estrogen deficient or deprivation state in mammals, in particular osteoporosis, bone loss, bone formation, cardiovascular effects more particularly hyperlipidaemia, prevention or the treatment of estrogen dependent or estrogen independent cancers such as cancer of breast and control or regulation of fertility in humans and in other animals. Detailed procedures for the evaluation of the compounds of the present invention or pharmaceutically acceptable salts or compositions thereof are described hereunder:

Test Procedure for Evaluation of Antiosteoporosis (Antiresorptive) Activity In Vitro

Test solutions of the compounds of the present invention are prepared in appropriate solvents in concentration range of 5 millimolar to 400 millimolar, most preferably in concentrations of 20 millimolar. 5 μl of each concentration are used for evaluation of antiresorptive activity in vitro. In control experiments, 5 μl of appropriate solvent is used in lieu of the test compound. Femur bones are isolated from chick embryos on day 11 post-ovulation. The adhering soft connective tissue is completely removed. Each femur bone is then placed in a drop of phosphate buffered saline (PBS) and is transferred to BGJ_{b} culture medium containing ⁴⁵CaCl₂ and incubated for 2 h. Labeled femur bones are washed 2-3 times with PBS and transferred to BGJ_{b} medium containing parathyroid hormone and cultured for 96 h in the presence or absence of the compound of invention or the vehicle in BGJ_{b} medium. Contralateral femur of each fetus serves as corresponding control. Culture medium with the respective treatment in each well is changed after 48 h. On termination of the culture at 96 h, bones are transferred to 0.1 N HCl for 24 h. Radioactivity due to ⁴⁵Ca in the spent medium collected at 48 and 96 h of culture and HCl extract at 96 h of culture is quantified by Liquid Scintillation Spectrophotometer in 10 ml of the scintillation fluid. Bone resorbing activity is expressed as percentage of ⁴⁵Ca released into the culture medium and the effect of the compound of invention as percent of the corresponding contra-lateral control or T/C ratio as shown below:

$$T / C ratio = \frac{{}^{45} Ca resorption in presence of PTH + test agent}{{}^{45} Ca resorption in presence of PTH + vehicle}$$

Appropriate solvents are selected from solvents like water, physiological saline, phosphate buffered saline, phosphate buffer, DMSO alone or in a suitable combination thereof.

In accordance with the above test procedure, the compounds of the present invention, on employing or administering their effective amounts, exhibit positive response by inhibiting parathyroid hormone (PTH) induced resorption of ⁴⁵Ca from chick fetal bones in culture. The compounds showing T/C ratio of ≦0.6 at ≦100 micromolar (μM) concentration are considered active (Table 1). Activity in the above test procedure indicates that the compounds of the present invention are useful as antiresorptive agents in the treatment of estrogen deficiency or deprivation (including post-menopausal) osteoporosis.

TABLE 1
Inhibition in PTH-induced resorption of ⁴⁵Ca from chick fetal
bones in culture

| Compound | Concentration in μM | T/C ratio |
|---|---|---|
| Example number 11 | 100 | 0.77 |
| Example number 12 | 100 | 0.78 |
| Example number 13 | 25 | 0.33 |
| | 50 | 0.41 |
| | 100 | 0.42 |
| | 200 | 0.61 |
| Example number 14 | 100 | 0.43 |
| Example number 15 | 100 | 0.54 |
| Example number 16 | 100 | 0.76 |
| Example number 18 | 100 | 1.60 |
| Example number 24 | 100 | 0.78 |
| Example number 35 | 100 | 0.99 |
| Example number 36 | 100 | 1.18 |
| Example number 39 | 100 | 0.85 |
| Example number 41 | 100 | 1.08 |
| Example number 49 | 100 | 0.63 |
| Example number 56 | 100 | 1.03 |
| Example number 58 | 100 | 0.98 |

Test Procedure for Evaluation of Antiosteoporosis Activity In Vivo

The in vivo antiosteoporosis activity is evaluated in colony-bred adult (3-4 month old) female Sprague-Dawley rats or female retired breeder Sprague-Dawley rats (12-14 months old; parity: ≧3). Animals are bilaterally ovariectomized (OVX) under light ether anesthesia and treated with the compound of the present invention, 17-alfa-ethynylestradiol (EE) or the vehicle once daily on days 1-30 post-ovariectomy (day 1: day of bilateral ovariectomy) by the oral route. One group of females is sham operated and treated similarly with the vehicle. Animals of all the groups are autopsied 24 h after the last treatment. Before autopsy, 24 h fasting urine samples are collected in fresh containers using all-glass metabolic cages and stored at −20° C. until analyzed for calcium, phosphorus and creatinine. At autopsy, about 5 ml blood samples are collected by cardiac puncture from each rat under light ether anesthesia and serum is isolated and stored at −20° C. until analyzed for total and bone specific alkaline phosphatase, osteocalcin and calcium. Uterus of each rat is carefully excised, gently blotted, weighed and fixed for histology. Representative sections (5 μm) from each uterus are stained with haematoxylin and eosin. Femur and tibia of each rat are then dissected free of adhering tissue, fixed in 70% ethanol in physiological saline and stored at −20° C. until Bone Mineral Density (BMD) measurements. Before autopsy, whole body scan of each rat for measurement of BMD is performed on an Hologic QDR-4500A fan-beam densitometer, calibrated daily with Hologic hydroxyapatite anthropomorphic spine phantom using manufacturer provided software for small animals. BMD measurement of isolated bones is performed using identical regions of interest. Serum total alkaline phosphatase, osteocalcin, calcium ion content and urinary calcium and creatinine are estimated colorimetrically using commercial kits.

Test Procedure for Evaluation of Antiproliferative/Cytotoxic Activity In Vitro

The procedure is based on the following methods: New calorimetric assay for anticancer drug screening, Skehan et al., J Natn Cancer Inst, 82,1107, 1990 and Feasibility of a high-flux anticancer drug screen using a diverse panel of cultured human tumor cell lines, J Natn Cancer Inst, 83,757, 1991.

A fully confluent flask of MCF-7 cells in trypsinized and 10⁴ cells/well are plated in a 96 welled flat bottomed plate in 200 μl Minimum Essential Medium (MEM), pH 7.4 and allowed to attach for 24 h at 37° C. in a humidified CO₂ incubator. Subsequently, the compound of invention dissolved in DMSO or ethanol is added at a specified concentration and further incubated for 48 h as before. The cells are then fixed in 50 μl cold 50% TCA and incubated for 1 h at 4° C. The supernatant is discarded and the plate is washed five times with deionized water and air-dried. 100 μl of 0.4% (w/v) Sulforhodamine B (SRB) in 1% acetic acid is added to each well and incubated at room temperature for 30 minutes. Unbound SRB is removed by five washes with chilled 1% acetic acid and the plate is air-dried. 200 μl of unbuffered 10 mM Tris base is added to solublize the bound stain for 5 minutes at room temperature and O.D. is read at 560 nm in a plate reader. The graph is plotted between O.D. and concentration and LC₅₀ is calculated with respect to tamoxifen, which is used as a positive control.

TABLE 2A
Antiproliferative/cytotoxic activity in cultured MCF-7 cells

| | |
|---|---|
| Compound | LC₅₀ (μM) |

(Test-1)

| | |
|---|---|
| Tamoxifen | 18.50 |
| Example number 7 | 46.00 |
| Example number 12 | 63.00 |
| Example number 13 | 24.00 |
| Example number 18 | 18.20 |
| Example number 51 | 22.30 |

(Test-2)

| | |
|---|---|
| Tamoxifen | 5.50 |
| Example number 9 | 37.44 |
| Example number 14 | 2.88 |
| Example number 25 | 15.04 |

(Test-3)

| | |
|---|---|
| Tamoxifen | 12.42 |
| Example number 18 | 9.63 |
| Example number 51 | 12.83 |

(Test-4)

| | |
|---|---|
| Tamoxifen | 9.80-10.00 |
| Example number 11 | 8.30 |
| Example number 22 | 19.50 |
| Example number 23 | 60.00 |
| Example number 24 | 27.00 |
| Example number 32 | 28.00 |
| Example number 33 | 18.20 |
| Example number 35 | 40.00 |
| Example number 36 | 60.00 |
| Example number 60 | 18.40 |

Test Procedure for Evaluation of Post-Coital Antifertility Activity

Adult female rats are caged overnight with coeval males of proven fertility and their vaginal smears are checked on the following morning. The day of presence of spermatozoa in the vaginal smear is taken as day 1 of pregnancy. Mated rats are isolated and randomized into various treatment groups and treated orally with the compound of invention or the vehicle on days 1-7/1-5 post-coitum. Animals of all the groups are autopsied on day 10 post-coitum and number and status of corpora lutea and implantation sites in each rat are recorded. The compounds of the present invention are considered active if there is complete absence of implantations in the uterus of all rats, in comparison to presence of normal implantations in the uterus of rats of vehicle control group.

TABLE 3
Post-coital antifertility efficacy in adult female rats

| | Daily dose | Treatment schedule | |
|---|---|---|---|
| Compound | (mg/kg) | (days post-coitum) | % Efficacy |
| Vehicle | — | 1-7 | — |
| Example number 5 | 10 | 1-7 | Inactive |
| Example number 7 | 10 | 1-7 | Inactive |
| Example number 11 | 10 | 1-7 | 50% |
| Example number 12 | 10 | 1-7 | Inactive |
| Example number 13 | 10 | 1-7 | Inactive |
| Example number 18 | 10 | 1-7 | Inactive |
| Example number 24 | 10 | 1-7 | Inactive |
| Example number 29 | 10 | 1-7 | Inactive |
| Example number 30 | 10 | 1-7 | Inactive |
| Example number 35 | 10 | 1-7 | Inactive |
| Example number 39 | 10 | 1-7 | Inactive |
| Example number 41 | 10 | 1-7 | Inactive |
| Example number 43 | 10 | 1-7 | Inactive |
| Example number 45 | 10 | 1-7 | Inactive |
| Example number 47 | 10 | 1-7 | Inactive |
| Example number 49 | 10 | 1-7 | 100% |
| | 5 | 1-5 | Inactive |
| Example number 51 | 10 | 1-7 | Inactive |
| Example number 56 | 10 | 1-7 | Inactive |
| Example number 58 | 10 | 1-7 | Inactive |
| Example number 60 | 10 | 1-7 | Inactive |

Test Procedure for Evaluation of Estrogen Agonistic Activity

Twenty-one-day-old immature female rats are bilaterally ovariectomized under light ether anaesthesia and, after post-operative rest for 7 days, are randomized into different treatment groups. Each rat receives the compound of the invention once daily for 3 consecutive days on days 28-30 of age. A separate group of animals receiving only the vehicle for similar duration serves as control. A autopsy 24 h after the last treatment on day 31 of age, vaginal smear of each rat is taken and uterus is carefully exised, gently blotted, weighed and fixed for histology and histomorphometry using image analysis. Premature opening of vagina, cornification of vaginal epitelium, increase in uterine fresh weight, total uterine and endometrial area and uterine luminal epithelial cell height are taken as parameters for evaluation of estrogen agonistic activity in comparision to rats of vehicle control group.

TABLE 4
Increase in uterine weight in immature ovariectomized rats:
Percent of ovariectomized + vehicle treatment group

| | Daily dose | |
|---|---|---|
| Compound | (mg/kg) | Percent increase |
| 17α-Ethynylestradiol | 0.01 | 419 |
| Example number 7 | 3 | 24.6 |
| | 10 | 108.7 |
| Example number 11 | 10 | 50.00 |
| Example number 12 | 3 | 3.40 |
| Example number 13 | 1 | 8.00 |
| | 3 | 2.53 |
| | 10 | 126.00 |
| Example number 14 | 1 | 41.00 |
| | 3 | 92.00 |
| | 10 | 144.00 |
| Example number 15 | 1 | 11.00 |
| | 3 | 15.00 |
| | 10 | 11.50 |
| Example number 16 | 10 | 2.00 |
| Example number 18 | 3 | 1.76 |
| | 10 | 51.00 |
| Example number 39 | 10 | 83.28 |
| Example number 41 | 10 | 42.85 |
| Example number 47 | 10 | 8.31 |
| Example number 51 | 3 | 21.88 |
| | 10 | 20.00 |
| Example number 56 | 10 | 15.42 |
| Example number 58 | 10 | 7.85 |

For image analysis in estrogen agonistic activity evaluation studies, paraffin sections (6 μm thick) of the uterus stained with haematoxylin and eosin are analysed microscopically. To determine changes in uterine tissue components, areas of the whole uterus and the endometrium and the thickness of uterine luminal epithelium are measured using a computer-image analysis system (BioVis, Expert Vision, India). Briefly, microscopic images of uterus acquired through a CCD camera are loaded in to the image analysis program and spatially calibrated against a stage micrometer image taken at the same magnification. Using thresholding and line tools, the regions for measurements are selected and the area (mm²) of whole uterine transection excluding the luminal space, the area (mm²) of the endometrium only, and the thickness (μm) of luminal epithelial lining are measured. Average of measurements made at 6 randomly selected sites are taken as parameters for evaluation of estrogen agonistic activity in comparison to rats of vehicle control group.
Test Procedure for Evaluation of Estrogen Antagonistic Activity

Twenty-one-day-old immature female rats are bilaterally ovariectomized under light ether anaesthesia and after post-operative rest for 7 days, are randomized into different treatment groups. Each rat receives the compounds of the invention and 0.02 mg/kg dose of 17-alfa-ethynylestradiol in 10% ethanol-distilled water once daily for 3 consecutive days on days 28-30 of age. A separate group of animals receiving only 17-alfa-ethynylestradiol (0.02 mg/kg) in 10% ethanol-distilled water for similar duration are used for comparison. At autopsy on day 31 of age, vaginal smear of each rat is taken and uterus is carefully excised, gently blotted, weighed and fixed for histology and histomorphometry using image analysis. Inhibition in 17-alfa-ethynylestradiol-induced premature opening of vagina, cornification of vaginal epithelium, increase in uterine fresh weight, total uterine and endometrial area and uterine luminal epithelial cell height are taken as parameters for evaluation of estrogen antagonistic activity.

TABLE 5
Percent inhibition in 17α-ethynylestradiol induced uterine weight
gain in immature ovariectomized rats

| | Daily dose | Percent |
|---|---|---|
| Compound | (mg/kg) | inhibition |
| Example number 7 | 10 | 15.45 |
| Example number 11 | 10 | 2.00 |
| Example number 13 | 1 | 12.00 |
| | 10 | 37.00 |
| Example number 14 | 1 | 3.00 |
| | 3 | 1.00 |
| | 10 | 29.00 |
| Example number 15 | 1 | 10.00 |
| | 3 | 23.00 |
| | 10 | 15.00 |
| Example number 16 | 10 | 2.00 |
| Example number 18 | 10 | 19.00 |
| Example number 39 | 10 | 3.70 |
| Example number 41 | 10 | 6.41 |
| Example number 47 | 10 | 17.2 |
| Example number 51 | 10 | 14.00 |
| Example number 56 | 10 | 12.83 |
| Example number 58 | 10 | 11.41 |

Test Procedure for Evaluation of Relative Binding Affinity (RBA) to Estrogen Receptors

The relative binding affinity (RBA) of the compounds for estrogen receptor is determined by competition assay, employing ³H-estradiol (³H-E₂) as the radioligand. The test ligands and ³H-E₂ are incubated at 4° C. with cystol estrogen receptors obtained from uteri of immature estradoil-primed (1 μg/rat 24 h before autopsy) 20-21 days old rats. Aliquot of uterine cytosol (200 μl; 2 uteri per ml) prepared in TEA buffer (10 mM Tris, 1.5 mM EDTA, 0.02% sodium azide, pH 7.4) are incubated in duplicate with a fixed concentration of ³H-E₂ in the absence or presence of various concentrations of the competitor substance dissolved in 30 μl of the TEA buffer containing DMF as co-solvent (final concentration of DMF in the incubation mixture never exceeded 5%) for 18 hrs at 4° C. At the end of this period, dextran coated charcoal (5% Norit 0.5% dextran) suspension in 100 μl of TEA buffer is added to each tube, briefly vortexed and allowed to stand for 15 minutes at 4° C. The mixture is centrifuged at 800 g for 10 minutes and the supernatants counted for radioactivity in 10 ml of a dioxane-based scintillation fluid. RBA of the text compound is computed from a graph plotted between percent bound radioactivity verses log concentration of the test substance. At 50% inhibition, log of the competitor concentration relative to that of 17-beta-estradiol, gives the affinity of the test compound to estrogen receptor relative to estradiol. This when multiplied with 100 gives the percentage value designated as RBA.

TABLE 6
Relative binding affinity (RBA) to estrogen receptors

| Compound | RBA |
|---|---|
| 17β-Estradiol | 100 |
| Example number 12 | 0.88 |
| Example number 13 | <0.001 |
| Example number 14 | <0.001 |
| Example number 15 | 0.059 |
| Example number 16 | <0.001 |
| Example number 18 | 0.17 |
| Example number 19 | 0.053 |
| Example number 20 | 0.10 |
| Example number 22 | <0.001 |
| Example number 23 | <0.001 |
| Example number 24 | 10.75 |
| Example number 25 | 0.83 |
| Example number 29 | <0.001 |
| Example number 30 | <0.001 |
| Example number 32 | <0.001 |
| Example number 33 | <0.001 |
| Example number 34 | 0.032 |
| Example number 35 | <0.001 |
| Example number 36 | <0.001 |
| Example number 37 | 0.36 |
| Example number 51 | 0.059 |

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. A compound useful for estrogen receptor disorders according to structural formula I, 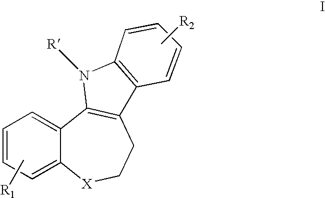
wherein
X is —O—, —S— or CH₂;
R′ is Y—(CH₂)ₙ or Y—(CH₂)ₙ—O-Ph, wherein n is 2 through 6, and wherein Y is selected from the moiety —NR₃R₄ wherein R₃ and R₄ are independently selected from a group consisting of pyrrolidinoethyl, piperidinoethyl, dimethylaminoethyl diethylaminoethyl and C₃-C₇ cycloalkyl;
a five membered saturated, unsaturated or partially unsaturated heterocycle containing up to two heteroatoms selected from the groups consisting of —O—, —NH—, —N(C₁C₄ alkyl)-, N═ and —S(O)ₘ, wherein m is an integer of from 0-2, optionally substituted with 1-3 substituents independently selected from the group consisting of H, OH, halo, nitro, cyano, SH, SO₂R₁, CO₂H, CONHR₁, NH₂;
a six membered saturated, unsaturated or partially unsaturated heterocycle containing up to two heteroatoms selected from the groups consisting of —O—, —NH—, —N(C₁C₄ alkyl, N═ and —S(O)ₘ, wherein m is an integer of from 0-2, optionally substituted with 1-3 substituents independently selected from the group consisting of H, OH, halo, nitro, cyano, SH, SO₂R₁, CO₂H, CONHR₁, NH₂;
a seven membered saturated, unsaturated or partially unsaturated heterocycle containing up to two heteroatoms selected from the groups consisting of —O—, —NH—, —N(C₁C₄ alkyl)-, N═ and —S(O)ₘ, wherein m is an integer of from 0-2, optionally substituted with 1-3 substituents independently selected from the group consisting of H, OH, halo, nitro, cyano, SH, SO₂R₁, CO₂H, CONHR₁, NH₂; a bicyclic heterocyclic containing from 6-12 carbon atoms either bridged or fused and containing up to two heteroatoms selected from the groups consisting of —O—, —NH—, —N(C₁C₄ alkyl)-, N═ and —S(O)ₘ, wherein m is an integer of from 0-2, optionally substituted with 1-3 substituents independently selected from the group consisting of H, OH, halo, nitro, cyano, SH, SO₂R₁, CO₂H, CONHR₁ and NH₂;
R₁ and R₂ are independently H, OH, —O(C₁-C₆ alkyl), —OCOC₆, H5, —OCO(C₁-C₆ alkyl), OSO₂(C₄C₆alkyl), —OSO₂CF₃, Cl or F; or a pharmaceutically acceptable salt thereof.

2. The compound of claim 1, wherein R₁ and R₂ are independently selected from H, OH, OCH₃.

3. The compound of claim 1, wherein n is 5 or 6.

4. The compound of claim 1, wherein Y is selected from acyclic or cyclic 5 or 6 membered saturated heterocyclic amine, preferably piperidine, pyrrolidine, N-methylbutylamine and the like.

5. The compound of claim 1, wherein X is S.

6. The compound of claim 1, wherein, Y is N-methylbutylamine or piperidine.

7. The compound of claim 1, wherein the compound is selected from the group consisting of:
12-[2-(piperidin-1-yl)ethyl]-6,7-dihydro-12H-benzothiepino [5,4-b]indole;
12-[2-(pyrolidin-1-yl ethyl]-6,7-dihydro-12H-benzothiepino [5,4-b]indole;
12-[2-(N-butyl methyl amino)ethyl]-6,7-dihydro-12H-benzothiepino [5,4-b]indole;
12-[3-(piperidin-1-yl)propyl]-6,7-dihydro-12H-benzothiepino [5,4-b]indole;
12-[3-(pyrrolidin-1-yl)propyl]-6,7-dihydro-12H-benzothiepino [5,4-b]indole;
12-[3-(N-butyl methyl amino)propyl]-6,7-dihydro-12H-benzothiepino [5,4-b]indole;
12-[4-(piperidin-1-yl)butyl]-6,7-dihydro-12H-benzothiepino [5,4-b]indole;
12-[4-(pyrrolidin-1-yl)butyl]-6,7-dihydro-12H-benzothiepino [5,4-b]indole;
12-[4-(N-butyl methyl amino)butyl]-6,7-dihydro-12H-benzothiepino [5,4-b]indole;
12-[4-(morpholine-4-yl)butyl]-6,7-dihydro-12H-benzothiepino [5,4-b]indole;
12-[5-(piperidin-1-yl)pentyl]-6,7-dihydro-12H-benzothiepino [5,4-b]indole;
12-[5-(pyrrolidin-1-yl)pentyl]-6,7-dihydro-12H-benzothiepino [5,4-b]indole;
12-[5-(N-butyl methyl amino)pentyl]-6,7-dihydro-12H-benzothiepino [5,4-b]indole;
12-[5-(N-butyl methyl amino pentyl]-6,7-dihydro-12H-benzothiepino [5,4-b]indole methyl iodide salt;
12-[5-(N-butyl methyl amino pentyl]-6,7-dihydro-12H-benzothiepino [5,4-b]indole ascorbic acid salt;
12-[5-(N-butyl methyl amino pentyl]-6,7-dihydro-12H-benzothiepino [5,4-b]indole fumaric acid salt;
12-[5-(morpholin-4-yl)pentyl]-6,7-dihydro-12H-benzothiepino [5,4-b]indole;
9-Methoxy-12-[5-(piperidin-1-yl)pentyl]-6,7-dihydro-12H-benzothiepino[5,4-b]indole;
9-Methoxy-12-[5-(pyrrolidin-1-yl)pentyl]-6,7-dihydro-12H-benzothiepino[5,4-b]indole;
9-Methoxy-12-[5-(N-butyl methyl amino)pentyl]-6,7-dihydro-12H-benzothiepino[5,4-b]indole;
9-Hydroxy-12-[5-(piperidin-1-yl)pentyl]-6,7-dihydro-12H-benzothiepino[5,4-b]indole;
9-Hydroxy-12-[5-(pyrrolidin-1-yl)pentyl]-6,7-dihydro-12H-benzothiepino[5,4-b]indole;
3-Hydroxy-12-[5-(N-butyl methyl amino)pentyl]-6,7-dihydro-12H-benzothiepino[5,4-b]indole;
3-Methoxy-12-[5-(piperidin-1-yl)pentyl]-6,7-dihydro-12H-benzothiepino[5,4-b]indole;
3-Methoxy-12-[5-(pyrrolidin-1-yl)pentyl]-6,7-dihydro-12H-benzothiepino[5,4-b]indole;
3-Methoxy-12-[5-(N-butyl methyl amino)pentyl]-6,7-dihydro-12H-benzothiepino[5,4-b]indole;
3-Hydroxy-12-[5-(piperidin-1-yl)pentyl]-6,7-dihydro-12H-benzothiepino[5,4-b]indole;
3-Hydroxy-12-[5-(pyrrolidin-1-yl)pentyl]-6,7-dihydro-12H-benzothiepino[5,4-b]indole;
3,9-Dimethoxy-12-[5-(piperidin-1-yl)pentyl]-6,7-dihydro-12H-benzothiepino[5,4-b]indole;
3,9-Dimethoxy-12-[5-(pyrrolidin-1-yl)pentyl]-6,7-dihydro-12H-benzothiepino[5,4-b]indole;
3,9-Dimethoxy-12-[5-(N-butyl methyl amino)pentyl]-6,7-dihydro-12H-benzothiepino[5,4-b]indole;
3,9-Dihydroxy-12-[5-(piperidin-1-yl)pentyl]-6,7-dihydro-12H-benzothiepino [5,4-b]indole;
3,9-Dihydroxy-12-[5-(pyrrolidin-1-yl)pentyl]-6,7-dihydro-12H-benzothiepino[5,4-b]indole;
3,9-Dihydroxy-12-[5-(N-butyl methyl amino)pentyl]-6,7-dihydro-12H-benzothiepino[5,4-b]indole;
12-[6-(piperidin-1-yl)hexyl]-6,7-dihydro-12H-benzothiepino[5,4-b]indole;
12-[6-(pyrrolidin-1-yl)hexyl]-6,7-dihydro-12H-benzothiepino[5,4-b]indole;
12-[6-(N-butyl methyl amino)hexyl]-6,7-dihydro-12H-benzothiepino[5,4-b]indole;
12-[6-(morpholin-4-yl)hexyl]-6,7-dihydro-12H-benzothiepino[5,4-b]indole;
12-[4-(2-piperidin-1-yl-ethoxy)benzyl]-6,7-dihydro-12H-benzothiepino[5,4-b]indole;
12-[4-(2-pyrrolodin-1-yl-ethoxy)benzyl]-6,7-dihydro-12H-benzothiepino[5,4-b]indole;
12-[4-{2-(N-butyl methyl amino)-ethoxy}benzyl]-6,7-dihydro-12H-benzothiepino[5,4-b]indole;
12-[4-(3-piperidin-1-yl-propoxy)benzyl]-6,7-dihydro-12H-benzothiepino[5,4-b]indole;
12-[4-(3-pyrrolidin-1-yl-propoxy)benzyl]-6,7-dihydro-12H-benzothiepino[5,4-b]indole;
12-[4-{3-(N-butyl methyl amino-propoxy)benzy}]-6,7-dihydro-12H-benzothiepino[5,4-b]indole;
12-[4-(5-piperidin-1-yl-pentyloxy)benzyl]-6,7-dihydro-12H-benzothiepino[5,4-b]indole;
12-[4-(5-pyrrolidin-1-yl-pentyloxy)benzyl]-6,7-dihydro-12H-benzothiepino[5,4-b]indole;
12-[4-{5-(N-butyl methyl amino)-pentyloxy}benzyl]-6,7-dihydro-12H-benzothiepino[5,4-b]indole;
12-[2-(piperidin-1-yl)ethyl]-6,7-dihydro-12H-benzoxepino[5,4-b]indole;
12-[2-(pyrolidin-1-yl) ethyl]-6,7-dihydro-12H-benzoxepino[5,4-b]indole;
12-[2-(N-butyl methyl amino) ethyl]-6,7-dihydro-12H-benzoxepino[5,4-b]indole;
12-[3-(piperidin-1-yl) propyl]-6,7-dihydro-12H-benzoxepino[5,4-b]indole;
12-[3-(pyrrolidin-1-yl) propyl]-6,7-dihydro-12H-benzoxepino[5,4-b]indole;
12-[3-(N-butyl methyl amino) propyl]-6,7-dihydro-12H-benzoxepino[5,4-b]indole;
12-[5-(piperidin-1-yl) pentyl]-6,7-dihydro-12H-benzoxepino[5,4-b]indole;
12-[5-(pyrrolidin-1-yl) pentyl]-6,7-dihydro-12H-benzoxepino[5,4-b]indole;
12-[5-(N-butyl methyl amino) pentyl]-6,7-dihydro-12H-benzoxepino[5,4-b]indole; 3-Methoxy-12-[5-(piperidin-1-yl)pentyl]-6,7-dihydro-12H-benzoxepino[5,4-b]indole; 3-Methoxy-12-[5-(pyrrolidin-1-yl)pentyl]-6,7-dihydro-12H-benzoxepino[5,4-b]indole; 3-Methoxy-12-[5-(N-butyl methyl amino)pentyl]-6,7-dihydro-12H-benzoxepino[5,4-b]indole; 3,9-Dimethoxy-12-[5-(piperidin-1-yl)pentyl]-6,7-dihydro-12H-benzoxepino[5,4-b]indole; 3,9-Dimethoxy-12-[5-(pyrrolidin-1-yl)pentyl]-6,7-dihydro-12H-benzoxepino[5,4-b]indole; 3,9-Dimethoxy-12-[5-(N-butyl methyl amino)pentyl]-6,7-dihydro-12H-benzoxepino[5,4-b]indole;
12-[4-(2-piperidin-1-yl-ethoxy)benzyl]-6,7-dihydro-12H-benzoxepino[5,4-b]indole;
12-[4-(2-pyrrolodin-1-yl-ethoxy)benzyl]-6,7-dihydro-12H-benzoxepino[5,4-b]indole;
12-[4-{2-(N-butyl methyl amino)-ethoxy}benzyl]-6,7-dihydro-12H-benzoxepino[5,4-b]indole;
12-[4-(3-piperidin-1-yl-propoxy)benzyl]-6,7-dihydro-12H-benzoxepino[5,4-b]indole;
12-[4-(3-pyrrolidin-1-yl-propoxy)benzyl]-6,7-dihydro-12H-benzoxepino[5,4-b]indole;
12-[4-{3-(N-butyl methyl amino)-propoxy}benzyl]-6,7-dihydro-12H-benzoxepino[5,4-b]indole;
12-[4-(5-piperidin-1-yl-pentyloxy)benzyl]-6,7-dihydro-12H-benzoxepino[5,4-b]indole;
12-[4-(5-pyrrolidin-1-yl-pentyloxy)benzyl]-6,7-dihydro-12H-benzoxepino[5,4-b]indole;
12-[4-{5-(N-butyl methyl amino)-pentyloxy}benzyl]-6,7-dihydro-12H-benzoxepino[5,4-b]indole;
12-[5-(piperidine-1-yl)-pentyl)]-5,6,7, 12-tetrahydro-benzo[6,7]cyclohepta[1,2-b]indole;
12-[5-(pyrrolidine-1-yl)-pentyl)]-5,6,7, 12-tetrahydro-benzo[6,7]cyclohepta[1,2-b]indole; and
12-[5-(N-butyl methyl amino)-pentyl)]-5,6,7, 12-tetrahydro-benzo[6,7]cyclohepta[1,2-b]indole.

8. A process for the preparation of a compound of claim 1 following the following scheme, 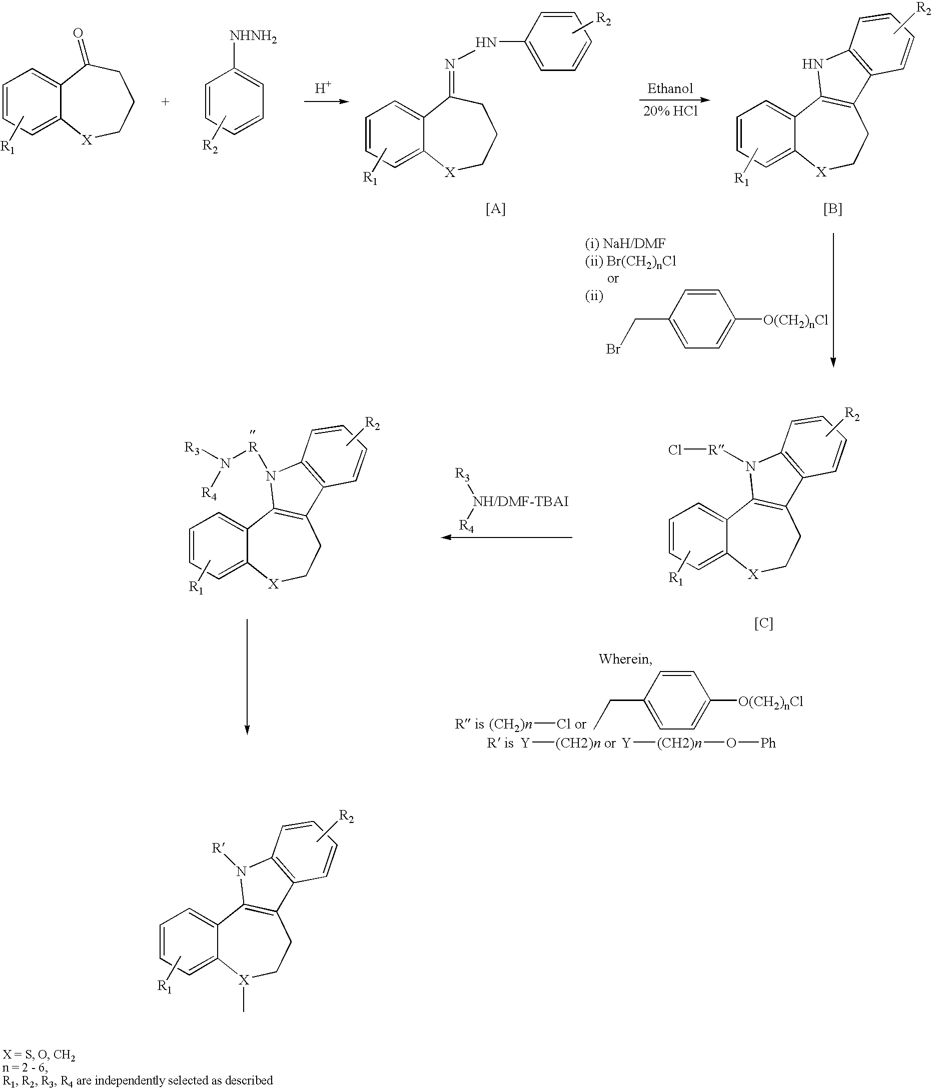
said process comprising:
(i) reacting a mixture of 3,4-dihydro-2H-benzo[b]thiepin-5-one or 3,4-dihydro-2H-benzo[b]oxepin-5-one or 6,7,8,9-tetrahydro-benzocyclohepten-5-one with substituted hydrazine and a protic acid for 4-5 hrs to form the compound of formula [A], wherein X is S, O or CH₂, and R₁ and R₂ are H;
(ii) reacting a mixture of the compound of formula [A] in ethanol and 15-30% aqueous hydrochloric acid for 12-15 hrs to form the compound of formula [B], wherein X is S, O or CH₂, and R₁ and R₂ are H;
(iii) reacting the compound of formula [B] with dihalo compounds in the presence of a suitable base in NaH in solvent DMF at O C under stirring conditions to form the compound of formula [C], wherein R″ is (CH₂)ₙ—Cl or -Ph-O—(CH₂)ₙ—Cl; X is S, O or CH₂ and n is 2 through 6; and
(iv) reacting the compound of formula [C] with cyclic or acyclic heteroamine in solvent DMF under stirring conditions at 70-75° C. in the presence of a catalyst to obtain the final compound of formula I, wherein X is S, O or CH₂; R₁ and R₂ is H and n is 2 through 6.

9. The process of claim 8, wherein the substituted hydrazine is selected from phenyl hydrazine and 4-methoxy phenyl hydrazine.

10. The process of claim 8, wherein said protic acid in step (i) is glacial acetic acid.

11. The process of claim 8, wherein said dihalo compounds in step (iii) are chlorobromoalkane or 4-(ψ-haloalkoxy) benzyl bromides.

12. The process of claim 8, wherein said cyclic or acyclic heteroamine in step (iv) is N-methylbutylamine or piperidine.

13. The process of claim 8, wherein said catalyst in step (iv) is tetrabutyl ammonium iodide.

14. A method for treating or preventing an estrogen related disease or syndrome in a subject in need thereof, comprising administering a pharmaceutically acceptable amount of a compound of claim 1 or a pharmaceutically acceptable derivative thereof.

15. The method of claim 14, wherein said estrogen related disease or syndrome is osteoporosis, bone loss, bone fracture, periodontal disease, metastatic bone disease, osteolytic bone disease, post plastic surgery, post-prosthetic joint surgery, or post dental implantation.

16. The method of claim 14, wherein said estrogen related disease or syndrome is caused by a cardiovascular disease, said cardiovascular disease comprising hyperlipidaemia, thrombosis, or vasomotor system or aortal smooth muscle cell proliferation.

17. The method of claim 14, wherein said estrogen related disease or syndrome is caused by a neurodegenerative disease, said neurodegenerative disease comprising stroke, senile dementia-Alzheimer's type, or Parkinson's disease.

18. The method of claim 14, wherein said estrogen related disease or syndrome is caused by menopausal symptoms, said menopausal symptoms comprising hot flushes, urogenital atrophy, depression, mania, schizophrenia and the like, urinary incontinence, relief of dysmenorrhea; relief of dysfunctional uterine bleeding, an aid in ovarian development, treatment of acne, or hirsutism.

19. The method of claim 14, wherein said estrogen related disease or syndrome is an estrogen dependent or estrogen independent cancer, said cancer comprising prostatic carcinoma, cancer of breast, cancer of uterus, cancer of the cervix, or cancer of the colon.

20. The method of claim 14 comprising an aid in ovarian development or function.

21. The method of claim 14 comprising the prevention or treatment of regulation of fertility in humans and in other animals.

22. The method of claim 14 comprising the prevention or treatment of threatened or habitual abortion.

23. The method of claim 14 comprising treatment of the suppression of post-partum lactation.

24. The method of claim 14 comprising prevention or treatment of a physiological disorder comprising obesity, depression and related disorders.

25. The method of claim 14 comprising the regulation of glucose metabolism in non-insulin dependent diabetes mellitus.

26. A pharmaceutical composition comprising a therapeutically effective amount of a compound of claim 1 or pharmaceutically acceptable salt thereof, and at least one pharmaceutically acceptable additive.

27. The pharmaceutical composition of claim 26, wherein said therapeutically effective amount is from about 0.01 mg to about 1000 mg.

28. The pharmaceutical composition of claim 26, wherein said therapeutically effective amount is from about 0.5 mg to about 500 mg.

29. The pharmaceutical composition of claim 26, wherein said therapeutically effective amount is from about 1.0 mg to about 100 mg.

30. The pharmaceutical composition of claim 26, wherein said therapeutically effective amount may be administered as a single dose or in multiple doses.
